(19) United States
(12) Patent Application Publication
(10) Pub. No.: US 2008/0213318 A1
(43) Pub. Date: Sep. 4, 2008

(54) Malaria MSP-1 C-terminal enhanced subunit vaccine

(75) Inventors: David E. Clements, Honolulu, HI (US); Tom Humphreys, Honolulu, HI (US)

Correspondence Address: GEORGE E. DARBY, P.O. BOX 893010, MILILANI, HI, 96789-3010, US

(73) Assignee: HAWAII BIOTECH, INC.

(21) Appl. No.: 11/481,709

(22) Filed: Jul. 3, 2006

Related U.S. Application Data
(60) Provisional application No. 60/696,895, filed on Jul. 5, 2005.
(60) Provisional application No. 60/814,375, filed on Jun. 16, 2006.

(51) Int. Cl. A61K 39/015; C12P 21/00; A61P 33/06

(52) U.S. Cl. 424/272.1; 435/69.1

(57) ABSTRACT

A vaccine or immunogenic composition is described that contains recombinantly produced, secreted, forms of malaria MSP-1 C-terminal subunit proteins from any of the Plasmodium falciparum strains as active ingredients combined with one or more adjuvants. The immunogenic compositions that result in a protective response are based on the use of a single adjuvant that forms an emulsion or use of such emulsion in combination with a second adjuvant that is an immunomodulating agent. Such a vaccine elicits a strong immune response characterized by antibodies that are capable of inhibiting parasite growth in vitro as well as antibodies that are incapable of inhibiting parasite growth in vitro, but are capable of enhancing the activity of the inhibitory antibodies. The disclosed vaccine formulations are capable of generating a protective response against malaria in vaccinated subjects.

### RELATED APPLICATIONS

This application claims the benefit of U.S. Provisional Patent Application No. 60/696,895, filed Jul. 5, 2005, and of U.S. Provisional Patent Application No. 60/814,375, filed Jun. 16, 2006, the disclosures and drawings of all of which prior applications are hereby incorporated by reference in their entirety.

### INCORPORATION OF SEQUENCE LISTING

A sequence listing file in ST.25 format on CD-ROM is appended to this application and fully incorporated herein by reference. The sequence listing information recorded in computer readable form is identical to the written sequence listing (per WIPO ST.25 para. 39, the information recorded on the form is identical to the written sequence listing). With respect to the appended CD-ROMs, the format is ISO 9660; the operating system compatibility is MS-Windows; the single file contained on each CD-ROM is named “MALp42ADJ03.ST25.txt” and is a text file produced by PatentIn 3.3 software; the file size in bytes is 21 KB; and the date of file creation is 30 Jun. 2006. The contents of the two CD-ROMs submitted herewith are identical.

### BACKGROUND OF THE INVENTION

1. Technical Field

The invention relates to vaccine formulations designed to protect against malaria. In particular, the vaccine formulations comprise recombinant subunit proteins derived from the C-terminal region of merozoite surface protein 1 (“MSP-1”) of Plasmodium falciparum. The largest of the C-terminal subunits is referred to as “p42”. The subunit proteins are produced in a cellular production system and, after purification, formulated in a vaccine with an adjuvant or adjuvant combination that generates an appropriate immune response. The vaccine formulations are shown to induce strong overall antibody titers as well as strong parasite growth inhibition antibodies in comparison to other formulations which produce weak parasite growth inhibition antibodies. Furthermore, the vaccine formulations are shown to provide protection against P. falciparum blood-stage infection in the Aotus monkey challenge model. These vaccine formulations have the potential to be used in humans to protect against malaria.

2. Related Art

The annual incidence of malaria is estimated to be 300 to 500 million cases, resulting in 1.5 to 3 million deaths annually (WHO, 1997). The majority of cases occur in Africa, although the threat extends to many other tropical and subtropical regions of the world. At this time there is a great need to control the spread of this disease since both the mosquito vector and the Plasmodium parasites have developed resistance to previous control measures. Over the past ten years, the primary focus has been on the development of malaria vaccines against the various developmental stages of the parasite. Although there has been considerable effort to establish technology for a recombinant subunit malaria vaccine and several candidate subunits have entered clinical trials, no vaccine is currently available. The term “parasites” herein means Plasmodium species unless otherwise specified.

There are several species of malaria parasites and each species has a defined host range. Plasmodium falciparum is the primary species that causes disease in humans. The life cycle of malaria parasites is complex. The parasite undergoes numerous developmental and morphological changes during the many stages of its life cycle. The cycle begins when an infected mosquito inoculates its host with sporozoites. The sporozoites quickly penetrate hepatocytes where they then develop into liver schizonts. Upon maturation, merozoites are released into the blood stream. The merozoites then invade erythrocytes where they multiply asexually until the infected cells burst, resulting in the release of additional merozoites that subsequently invade additional erythrocytes. The multiplication of the merozoites and the lysis of erythrocytes are associated with the clinical symptoms of malaria. Some merozoites go on to develop into male and female gametocytes which are then taken up by mosquitoes feeding on infected individuals. Once in the mosquito, fertilization of the female gamete by the male gamete leads to further development into the sporozoite stage of the parasite. Thus, the cycle is set to begin again. The life cycle is divided into three stages, pre-erythrocytic, asexual erythrocytic, and sexual stage. In regard to vaccine development, the three stages are often referred to as sporozoite or liver stage (pre-erythrocytic), blood stage (asexual erythrocytic), and transmission blocking (sexual stage).

While the potential exists for the development of malaria vaccines, the efforts of the past decade have proven that the development of such vaccines is a difficult task. Progress has been made in identifying and developing proteins as malaria vaccine candidates. The primary means of selecting proteins for use in malaria vaccines has been by screening for those which are reactive to human immune serum. In this way, a number of proteins in each of the life-cycle stages have been identified as vaccine candidates. The majority of these proteins that have been advanced as vaccine candidates are ones that are expressed on the surface of the parasite. Because of the difficulty in culturing parasites as a source of antigen, the development of malaria vaccines has had to rely on alternative methodologies such as recombinant DNA. This technology has been successfully utilized to define important peptide sequences, to express subunit antigens, and to develop DNA-based vaccines.

While there are efforts under way to develop malaria vaccines that target each of the developmental stages of the parasite, a vaccine targeting the blood stage is likely to have the greatest impact, since this is the stage that results in disease manifestation (Good et al, 1998). Of the many blood stage antigens that have been identified, the major merozoite surface protein (MSP-1) is one that has been extensively characterized.

The native MSP-1 protein has a molecular weight of approximately 195 kD. It is a membrane anchored molecule that is prominently displayed on the surface of merozoites. It is processed into four major fragments, which are referred to by their relative molecular weights, p83, p28, p38 and p42 (Hall et al, 1984, Lyon et al, 1986 and Holder et al 1987). The function of all of the fragments is not known. It has been determined that the C-terminal p42 fragment, which is anchored on the surface of the merozoite, is further processed to two fragments referred to as p33 and p19, and that this processing is a prerequisite for erythrocyte invasion (Blackman et al, 1990, 1991 and Blackman and Holder, 1992).

A number of findings support MSP-1 as an important malaria vaccine candidate. It has been demonstrated that monkeys can be protected from P. falciparum challenge when immunized with MSP-1 derived from cultured parasites (Hall et al 1984, Siddiqui et al 1987, and Etlinger et al 1991). Also, recombinant or synthetic peptides representing various portions of MSP-1 have also been shown to elicit various levels of protection in challenge studies (Hall et al, 1984, Cheung et al, 1986, Patarroyo et al, 1987, Herrera et al, 1990, Kumar et al, 1995, and Chang et al, 1996, Kumar et al, 2000, Stowers et al, 2001, Darko et al, 2005). In addition, analysis of serum collected from humans living in endemic areas has shown that the production of IgG antibodies directed at the C-terminus of MSP-1 correlates with the development of clinical immunity against falciparum malaria (Riley et al, 1992, Shai et al, 1995, Al-Yaman et al, 1996, and Shi et al, 1996). Lastly, it has been demonstrated that monoclonal antibodies against MSP-1 are capable of preventing parasite invasion of erythrocyte in vitro (Pirson and Perkins, 1985 and Blackman et al, 1990).

The C-terminal p42 fragment and its p19 processed fragment have been identified as leading subunit vaccine candidates derived from the MSP-1 protein. The sequence of the p19 region is highly conserved among different P. falciparum strains. The p19 region also contains 6 disulfide bridges that result in a highly folded structure that represents two epidermal growth factor (EGF)-like domains (Blackman et al, 1991). Both polyclonal and monoclonal antibodies directed at p19 and p42 subunits have been shown to inhibit parasite development in vitro (Blackman et al, 1990, Chang et al, 1992, Chappel and Holder, 1993).

Efforts to develop a recombinant subunit vaccine based on the p42 or p19 fragments of MSP-1 have utilized several different heterologous protein expression systems. The expression of these subunits in E. coli, Saccharomyces, and baculovirus vector systems is summarized below. Antigens expressed in E. coli induce only low levels of antibodies reactive to native protein upon immunization (Holder et al, 1988, Burghaus et al, 1996). Therefore, most efforts are currently focused on expression systems that have the capability of directing the authentic folding of the MSP-1 subunits in an attempt to produce a more antigenically relevant product.

Kumar et al (1995) were able to demonstrate protection of Aotus monkeys when immunized with a p19 subunit that was expressed in yeast. While protection was achieved in some monkeys with the yeast-derived p19, serum from these monkeys was not able to inhibit parasite growth in vitro. Based on these results, it was suggested that specific inhibiting antibodies (those antibodies which are capable of inhibiting parasite growth in vitro) where not the only factor that contributed to protection upon immunization with a recombinant MSP-1 C-terminal subunit. Utilizing the baculovirus system, Chang et al (1996) were able to produce a p42 product that is highly immunogenic in Aotus monkeys and confers protection against malarial challenge. Unlike the yeast-expressed p19, the serum from monkeys protected by the baculovirus-expressed p42 was able to inhibit the in vitro growth of parasites. From these results, it was suggested that parasite growth inhibition is a useful correlate of protective immunity.

Despite the success achieved with MSP-1 C-terminal subunits thus far, there are still three key issues regarding the use of these subunits as vaccine candidates that require further investigation. The first involves identifying the relevant immunogenic segments of the MSP-1 C-terminal region in regards to eliciting specific protective antibody responses. Specific protective antibodies are those which are capable of inhibiting parasite growth in vitro. It is known that there are also antibodies that are not capable of inhibiting parasite growth in vitro, but can contribute to the overall protective response (non-specific protective antibodies). Therefore, a second area of investigation is the identification of relevant immunogenic segments responsible for eliciting non-specific protective antibodies, called herein “enhancing antibodies”, that contribute to the overall protective response upon immunization The third area of investigation involves the identification of clinically relevant adjuvants that are capable of generating an appropriate immune response, i.e., the induction of protective antibody responses.

As indicated by the attempts described above to generate protective responses in animal models, it is not clear which segments of the MSP-1 C-terminal region (defined as p42 and subunits of p42) are most relevant in generating a protective immune response. The data suggest that all of p42, or possibly a portion of the p33 region of p42, contributes to improved immunogenicity (“enhancing antibodies”). Also, the heterologous expression system used to produce the recombinant subunit proteins will most likely have an impact on the quality of the product expressed and thus impact the quality of subsequent immune responses.

It is important that an expression system be able to produce both a high quality product and high yields of the desired product. In an effort to meet these criteria, the Drosophila expression system, as defined below, was selected by the inventors for the expression of MSP-1 C-terminal subunits. This system has been shown to be able to express heterologous proteins that maintain native-like biological structure and function (Bin et al, 1996 and Incardona and Rosenberry, 1996). The Drosophila expression system is also capable of producing high yields of product. The use of an efficient expression system will ultimately lower the cost per dose of a vaccine and enhance the commercial potential of the product.

The inability to generate solid protective responses in primate trials with an adjuvant other than Freund's Complete Adjuvant (FCA) has been a major hurdle in the development of P. falciparum MSP-1 subunit vaccines. Since FCA is not an adjuvant suitable for use in humans, there is a need to identify an alternate formulation capable of potentiating a protective response. In an early study with MSP-1 purified from parasites, the protective ability of a formulation with muramyl dipeptide (MDP) was compared to a formulation with FCA (Siddiqui et al, 1986). While both formulations elicited high anti-MSP-1 antibody titers, only the FCA formulation resulted in protection. These results established that the selection of an appropriate adjuvant is essential for generating a protective response to MSP-1. Although this was a limited study, the conclusion, that a “strong” adjuvant, such as FCA, is needed to elicit a protective response, is widely held. However, the nature of this requirement has yet to be clearly defined.

The further development of an MSP-1 C-terminal subunit, or any malaria subunit vaccine, critically depends on identifying a clinically relevant adjuvant. Modern adjuvant research has made significant advances toward producing a variety of adjuvants that have properties that influence the nature of the immune response that they generate with specific antigens. Some of these adjuvants focus more on antibody responses and others are known to stimulate cellular responses as well. In addition, different subsets of antibody isotypes and subclasses are induced by individual adjuvant-antigen formulations. Although not all of the parameters of a protective response induced by blood stage antigens are known, present adjuvant technology offers a diversity of possibilities that can be tested empirically and used to define the nature of the protection conferred by MSP-1 C-terminal subunits.

In an effort to identify clinically relevant adjuvants that are capable of potentiating a protective response, the immune mechanisms relevant to the target antigen must be considered. Seroepidemiological studies have established a correlation between the presence of antibodies to the C-terminal region of MSP-1 and resistance to clinical malaria (Riley et al, 1992, Al-Yaman et al, 1996 and Egan et al, 1996). In a recent report, it was determined that the antibodies in naturally infected humans directed at the p19 region are mainly IgG1 (Cavanagh et al, 2001). In the malaria mouse model, protective monoclonal antibodies (“MAbs”) have been shown to be reactive to the C-terminal region of MSP-1 (Burns et al, 1988). Also, it has been demonstrated that immunization of mice with recombinant MSP-1 subunits results in protection from lethal P. yoelii challenge (Daly and Long, 1996 and Hirunpetcharat et al, 1997). These studies determined that protection is dependent on the induction of appropriate antibody responses. Results in primates with recombinant MSP-1 C-terminal subunits from P. falciparum (Chang et al, 1996) and P. vivax (Perera et al, 1998) also establish the role of antibodies in protection from challenge. These results establish that antibodies directed at the C-terminal region of MSP-1 play a role in protection; however, the requirements for eliciting such antibody responses with subunit proteins and adjuvants are not well defined. The difficulty of identifying appropriate adjuvants is demonstrated in a study conducted by Kumar et al (2000). In this study, FCA was the only adjuvant of several that were tested that resulted in a protective response when formulated with the p19 subunit from yeast. Thus, it is still necessary that systematic approaches be applied to define the nature of protein subunits utilized and in choosing the appropriate adjuvant or combination of adjuvants.

Other attempts to identify alternative adjuvants have been conducted in both mouse and monkey challenge models. There are two reports where strong protective responses have been elicited with modern adjuvants in mice immunized with P. yoelii MSP-1 subunits (De Souza et al, 1996 and Ling et al, 1997). In a study by Ling et al (1997), an MPL/QS21 adjuvant formulation (SBAS2) was used. The success of eliciting a protective response in the monkey model with modern adjuvants has been limited. In a protection study with an MSP-1 p19 subunit from P. vivax formulated with Alum or block copolymer P1005 adjuvant, it was reported that the alum formulated antigen failed to induce a protective response and the P1005 formulation resulted in partial protection (Yang et al, 1999).

In the studies described above, most antigen/adjuvant combinations were capable of eliciting high overall antibody titers, despite the lack of a protective response. This was most notable in the study of Kumar et al (2000) and demonstrates the production of high titers of antibodies with specific antigen preparations does not always result in a protective response.

Calcium and aluminum salts are currently the only licensed adjuvants for use in human vaccine products. Numerous studies have demonstrated that other adjuvants are significantly more efficacious for inducing both humoral and cellular immune responses. However, most of these have significant toxicities or side-effects that make them unacceptable for human and veterinary vaccines. In fact, even aluminum hydroxide has recently been associated with the development of injection site granulomas in animals, raising safety concerns about its use. Because of these problems significant efforts have been invested in developing highly potent, but relatively non-toxic adjuvants. A number of such adjuvant formulations have been developed and show significant promise (Cox, J. C. and Coulter, A. R., Vaccine (1997) 15:248-256; Gupta, R. K. and Siber, G. R., Vaccine (1995) 13:1263-1276), especially in combination with recombinant products. Several of these modern adjuvants are being tested in preclinical and clinical trials designed to examine both efficacy and safety. The modes of action of adjuvants include: (i) a depot effect, (ii) immunomodulation, (iii) targeting specific antigen-presenting cell populations, (iv) formation of micelles or liposomes, and (v) maintaining appropriate “native” conformation of the antigen. The depot effect results from either the adsorption of protein antigens onto aluminum gels or the emulsification of aqueous antigens in water-in-oil formulations. In either case, upon immunization this results in the slow release of antigen into the circulation from local sites of deposition. This prevents the rapid loss of most of the antigen that would occur by passage of the circulating antigen through the liver. Immunomodulation involves stimulation of the “innate” immune system through interaction of particular adjuvants with cells such as monocytes/macrophages or natural killer (NK) cells. These cells become activated and elaborate proinflammatory cytokines such as TNF-alpha and IFN-gamma, which in turn stimulate T lymphocytes and activate the “adaptive” immune system. Bacterial cell products, such as lipopolysaccharides, cell wall derived material, DNA, or oligonucleotides often function in this manner (Krieg, A. M. et al., Nature (1995) 374:546; Ballas, Z, J, et al., J. of Immunology (2001) 167:4878-4886; Chu, R. S., et al., J. Exp. Med. (1997) 186:1623; Hartmann, G. and Krieg, A., J. Immunol. (2000) 164:944-952; Hartmann, G., et al., J. of Immunol. (2000) 164:1617-1624; Weeratna, R. D. et al., Vaccine (2000) 18:1755-1762; and U.S. Pat. Nos. 5,663,153; 5,723,335; 6,194,388; 6,207,646; 6,214,806; 6,218,371; 6,239,116; 6,339,068; 6,406,705; 6,429,199). Finally, some adjuvants may have the ability to maintain the antigen in its “native” conformation, thereby protecting important “conformational” epitopes. These epitopes may be important for eliciting the production of antibodies with particular functional capabilities, such as parasite growth inhibition.

The development of a viable malaria vaccine based on the use of MSP-1 C-terminal subunits requires the identification of clinically relevant adjuvants that can be used in combination with these subunits to induce overall high antibody titers and also induce specific parasite growth inhibition antibodies. Furthermore, the development of a viable malaria vaccine for MSP-1 C-terminal subunits requires the identification of an expression system that provides high yields of subunit proteins with native conformation. The combination of appropriate, clinically relevant adjuvants and native-like subunits to formulate a malaria vaccine such that a protective immune response in primate animal models and humans can be induced is the desired outcome. Therefore, the technical problems to be solved are: (1) identification and/or development of an expression system that provides high yields of subunit proteins with native-like conformation, (2) the identification of clinically relevant adjuvants that are capable of inducing the generation of specific parasite growth inhibition antibodies when combined with recombinant p42 subunits, and (3), formulation and administration of a vaccine containing such one or more adjuvants and subunit proteins that induces protective immunity against malaria in animal models and humans. Further improvement of malaria vaccines based on the MSP-1 C-terminal region could potentially be made through the identification of novel recombinant subunits that result in improved immunogenicity and protective responses. Therefore an additional technical problem to be solved is: the design, construction, and expression of novel subunits of the C-terminal region of the MSP-1 protein that result in improved immunogenicity and protective responses.

### SUMMARY OF THE INVENTION

The invention provides subunit proteins and immunogenic compositions that can be utilized as vaccines to protect against malaria in animal models and humans. The recombinant subunit proteins are expressed from transformed insect cells that contain integrated copies of the appropriate expression cassettes in their genome. The insect cell expression system provides high yields of recombinant subunit proteins with native-like conformation. Specifically, the recombinant subunit proteins are secreted from the transformed insect cells and represent truncated forms of the malaria merozoite surface protein, MSP-1. More specifically, the subunits are derived from the C-terminal region of the MSP-1 protein.

The invention also provides for the use of water in oil emulsion adjuvants alone or in combination with monophosphoryl lipid A derivates as components for the effective formulation of an immunogenic composition suitable for a malaria vaccine.

The invention also provides methods for utilizing the vaccines to elicit the production of antibodies capable of conferring protection against malaria in mammalian hosts. The protective antibodies can be either “inhibitory antibodies,” which are capable of inhibiting parasite growth in vitro, or “enhancing antibodies”, which are incapable of inhibiting parasite growth in vitro, but which still enhance the protection provided by inhibitory antibodies.

### BRIEF DESCRIPTION OF THE DRAWINGS

FIG. 1. Amino acid sequence of MSP-1 p42 (SEQ ID NO:1) from the P. falciparum strain FUP (Uganda Palo Alto) of the MAD type.

FIG. 2. Amino acid sequence of MSP-1 p42 (SEQ ID NO:2) from the P. falciparum strain 3D7 of the Wellcome type.

FIG. 3. Amino acid sequence of MSP-1 p42 (SEQ ID NO:3) from the P. falciparum strain FVO (Vietnam-Oak Noll) of the K1 type.

FIG. 4. Alignment of the MSP-1 p42 amino acid sequences from the three P. falciparum strains FUP, 3D7, and FVO. Amino acids differing from that in the FUP strain are in bold.

FIG. 5. Alignment of the amino acid (“aa”) sequences from three N-terminally truncated subunits, C31p19 (SEQ ID NO:7), C72p19 (SEQ ID NO:8), and CTC72p19 (SEQ ID NO:9). The variant amino acids in the truncated subunits of the 3D7 strain, compared with the counterpart amino acids in the C31p19 subunit of the FUP strain, are in bold and not underlined. The CT and C72 epitopes in the p42 sequence are in bold and underlined.

FIG. 6. Reactivity of S2 expressed MSP-1 p42 to a panel of monoclonal antibodies.

FIG. 7. Evaluation of MSP-1 p42 expressed in Drosophila. Culture medium samples from two S2 cell lines transformed with FUP MSP-1 p42 expression plasmids, pMttp42.3.7 and pMttp42.3.11. 10 microliters of unconcentrated culture medium from 5 ml of induced cultures are compared to known amounts of purified p42 as a means assessing the level of expression from S2 cells. Samples (non-reducing) were separated on a 10% SDS-PAGE gel and stained with Coomassie blue. Lane 1—MW markers 175, 83, 62, 47, 32, and 25 kD; lane 2—pMttp42.3.7; lane 3—pMttp42.3.11; lane 4—blank; lane 5—2 μg; lane 6—1 μg; lane 7—500 ng; lane 8—250 ng, The arrow indicates the position of p42.

FIG. 8. T-cell proliferation results for mouse splenocytes primed with one, two, or three doses of MSP-1 p42 and then stimulated with p33 peptides.

FIG. 9. Cytokine responses of p42 primed splenocytes stimulated with p33 peptides after one, two, and three doses of p42.

FIG. 10. Parasitemia in Aotus monkeys immunized with p42 and then challenged with P. falciparum FUP strain.

FIG. 11. Cumulative parasites counts in p42 immunized Aotus monkeys following challenge with P. falciparum FUP strain.

FIG. 12. Parasite growth inhibitory activity of anti-MSP-1 p42 serum from rabbit (Rbt) 13 in the presence of non-inhibitory anti-MSP-1 p42 sera.

FIG. 13. Parasite growth inhibitory activity of anti-MSP-1 p42 serum from rabbit (Rbt) 15 in the presence of non-inhibitory anti-MSP-1 p42 sera.

FIG. 14. Parasite growth inhibitory activity of anti-MSP-1 p42 serum from rabbit (Rbt) 16 in the presence of non-inhibitory anti-MSP-1 p42 sera.

### DETAILED DESCRIPTION OF THE INVENTION

The invention provides malaria MSP-1 recombinant subunit proteins that are produced and secreted from a stable insect cell lines that have been transformed with the appropriate expression plasmid and are combined with adjuvant(s) such that they are effective in inducing a strong inhibiting antibody response capable of inhibiting the growth of Plasmodium falciparum. The use of appropriate adjuvants or adjuvant combinations is critical for the induction of a specific immune response that results in antibodies that are capable of inhibiting parasite growth and ultimately providing protection form malaria.

In a preferred embodiment of the invention, the recombinant malaria subunit proteins that are a component of the vaccine formulation described herein are produced in a eukaryotic expression system which utilizes insect cells. Insect cells are an alternative eukaryotic expression system that provides the ability to express properly folded and post-translationally modified proteins while providing simple and relatively inexpensive growth conditions. The majority of insect cell expressions systems are based on the use of baculovirus-derived vectors to drive expression of recombinant proteins. Expression systems using baculovirus-derived vectors, however, are not stable: over-expression of the desired product by the baculovirus vector also results in virus production, which leads to lysis of the host cells. As a result, each production run that utilizes baculovirus vectors requires that the host cells be infected and then harvested after one generation of growth. Expression systems based on stable cell lines due to the integration of expression cassettes into the genome of the host cell are capable of being used over multiple generations for the expression of the desired product. This provides a greater level of consistency in the production of product. The Drosophila melanogaster expression system (“Drosophila expression system” or “Drosophila system”) (Johansen, H. et al., Genes Dev. (1989) 3:882-889; Ivey-Hoyle, M., Curr. Opin. Biotechnol. (1991) 2:704-707; Culp, J. S., et al., Biotechnology (NY) (1991) 9:173-177) is an insect cell expression system based on the generation of stably transformed cell lines for recombinant protein expression. This insect cell expression system has been shown to successfully produce a number of proteins from different sources. Most importantly, the recombinant proteins produced in this expression system have been shown to maintain structural and functional characteristics of the corresponding native proteins. Examples of proteins that have been successfully expressed in the Drosophila expression system include HIV gp120 (Culp, J. S., et al., Biotechnology (NY) (1991) 9:173-177; Ivey-Hoyle, M., Curr. Opin. Biotechnol. (1991) 2:704-707 and U.S. Pat. Nos. 6,165,477, 6,416,763, 6,432,411, and 6,749,857), human dopamine β-hydrolase (Bin et al, 1996), human vascular cell adhesion protein (Bernard et al, 1994), and dengue envelope glycoprotein (Modis et al, 2003). In each of these examples, expression levels were greater than other systems that had been utilized and, more importantly, the products expressed were of higher quality based on functional and/or structural studies.

While the Drosophila expression system has the potential to produce structurally and immunologically relevant proteins, not all attempts to express heterologous proteins or truncated versions of proteins have met with success. Therefore, a systematic evaluation is required to determine the potential to express a particular heterologous protein subunit in the Drosophila expression system. Examples of proteins and their subunits that have failed to express adequately in the Drosophila system include the dengue and hepatitis C NS3 proteins, subunits of the dengue NS1 protein, subunits of the dengue E protein, and subunits of the malaria LSA-1 protein. Most importantly, the malaria p19 subunit of MSP-1 protein, a truncated form of p42, which has been successfully expressed in yeast expression systems has been shown to be poorly expressed (to date) in the Drosophila expression system.

In the present invention, the expression and secretion of subunit proteins comprising MSP-1 p42 and N-terminally truncated forms of MSP-1 p42 (referred to hereafter as “MSP-1 p42 subunit” and “N-terminally truncated MSP-1 p42 subunit”, respectively, and collectively as “MSP-1 C-terminal subunits”) from stably transformed Drosophila S2 cells were evaluated by operably linking the coding sequences of such proteins to the tPA (tissue plasminogen activator) secretion signal and placing them under the control of the Drosophila MtnA (metalothionein) promoter utilizing standard recombinant DNA methods. The recombinant MSP-1 C-terminal subunits described herein are derived from multiple strains of Plasmodium falciparum. The sequences encoding the MSP-1 p42 subunits from the three strains of P. falciparum, i.e., FUP (Uganda Palo Alto) of the MAD type (SEQ ID NO:4), NF-54 (clone 3D7) of the Wellcome type (SEQ ID NO:5), and FVO (Vietnam-Oak Noll) of the K1 type (SEQ ID NO:6), were cloned into Drosophila expression plasmids and then used to transform Drosophila S2 cells. The nucleotide sequences, SEQ ID NOs:4, 5, and 5, encode the corresponding amino acid sequences, SEQ ID NOs:1, 2, and 3, respectively. The nucleotide sequences of SEQ ID NOs:4, 5, and 6 may have significant substitution, depending upon impact in secondary and tertiary structure of the protein encoded by a given nucleotide sequence and on the corresponding immunogenicity.

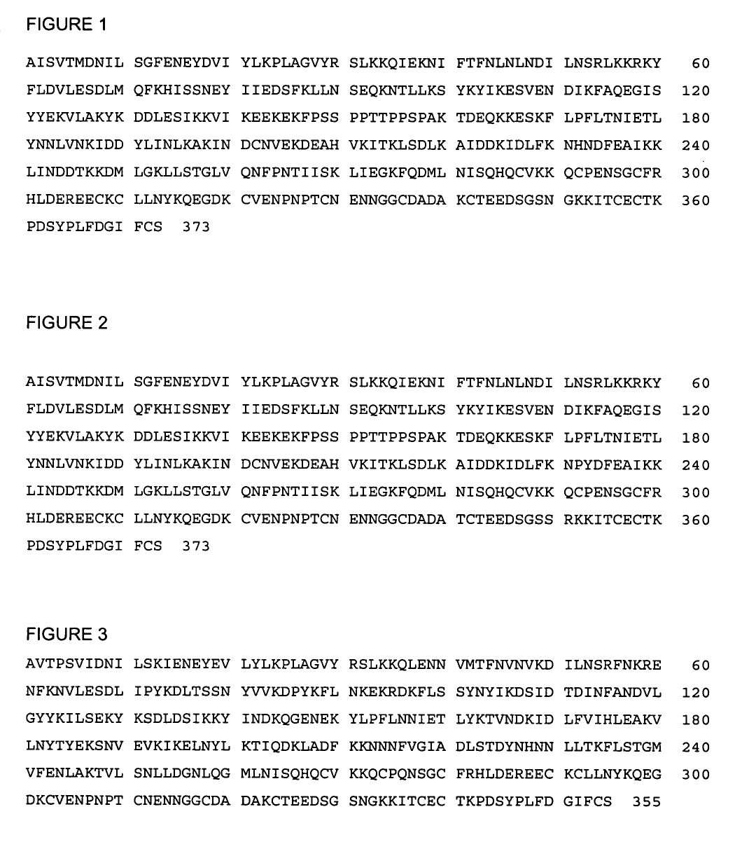

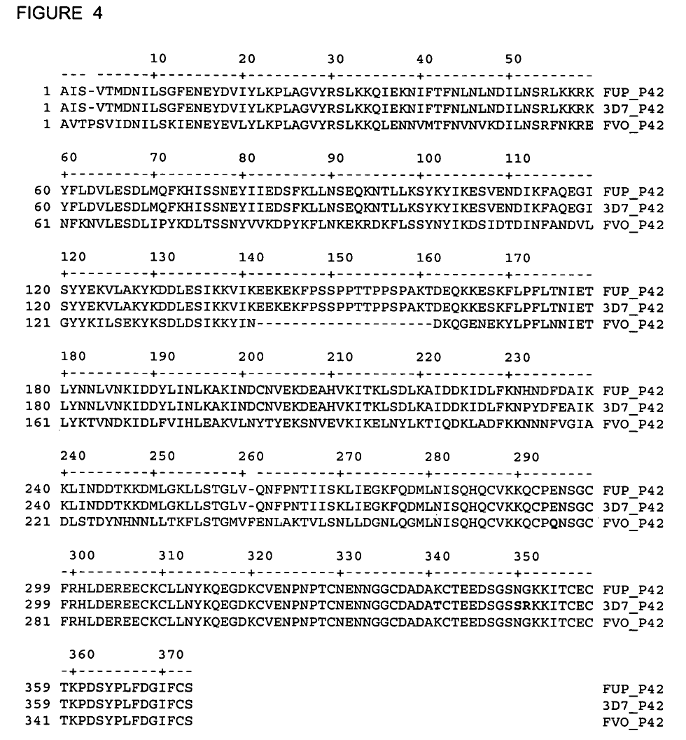

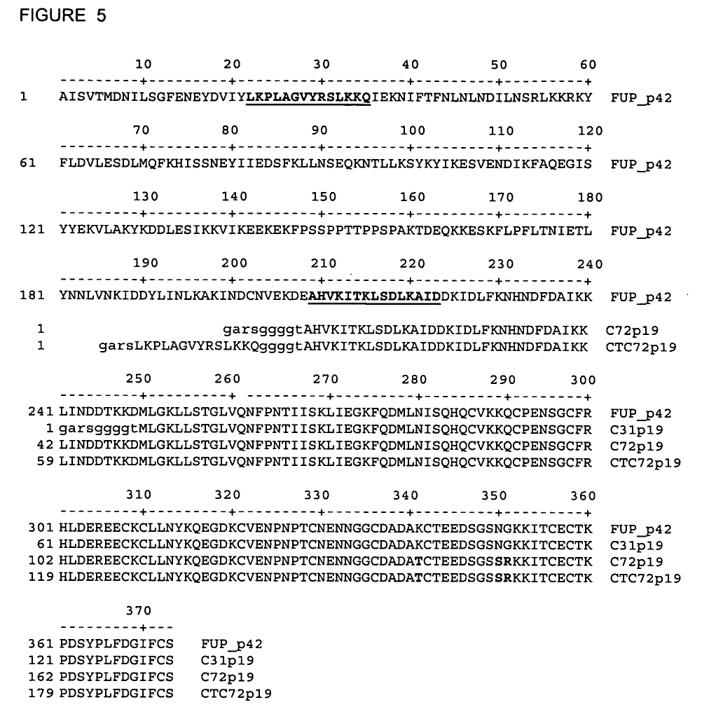

The amino acid sequence of the MSP-1 p42 subunits from the three strains of P. falciparum expressed in S2 cells are shown in FIGS. 1-3. FIG. 4 provides an alignment of the amino acid sequences of the MSP-1 p42 subunits from the three strains. The N-terminally truncated MSP-1 p42 subunits were derived from the cloned p42 sequences of the 3D7 and FUP strains. Three N-terminally truncated MSP-1 p42 subunits were constructed that had different truncation points and conserved sequences added. The first two subunits contained different lengths of sequence from the C-terminal region of p33 in continuum with the p19 region. These subunits are referred to by the number of p33 C-terminal amino acid residues that precede p19. Thus, the two subunits are referred to as C31p19 and C72p19. The C31p19 subunit has the amino acid sequence shown in SEQ ID NO:7. The C72p19 subunit has the amino acid sequence shown in SEQ ID NO:8. The third subunit, CTC72p19 (also called the “CT subunit” herein), is based on the C72p19 subunit and contains a conserved epitope from the N-terminal region of p33 (aa 23-35 in FIG. 5) fused to the N-terminus of C72p19, as more fully described in Example 1 below. The CTC72p19 subunit has the amino acid sequence shown in SEQ ID NO:9. The nucleotide sequences, SEQ ID NOs: 10, 11, and 12, encode the respective amino acid sequences, SEQ ID NOs: 7, 8, and 9. The three N-terminally truncated MSP-1 p42 subunits are shown in the alignment in FIG. 5. In regards to the extent of the N-terminal region that was removed to generate the N-terminally truncated MSP-1 p42 subunits, C31p19 and C72p19 subunits represent removal of 67% and 56% respectively. Alternatively, the C31p19 and the C72p19 subunits represent 33% and 44% of the carboxy-terminal end of p42 respectively (p19 itself represents 25% of the carboxy-terminal end of p42). The C72p19 and CTC72p19 subunits are based on the 3D7 sequence and the C31pl9 subunit is based on the FUP sequence. The amino acid sequences of SEQ ID NOs: 7, 8, and 9 may have as much as 10% substitution, depending upon impact in secondary and tertiary structure, and still retain some immunogenicity.

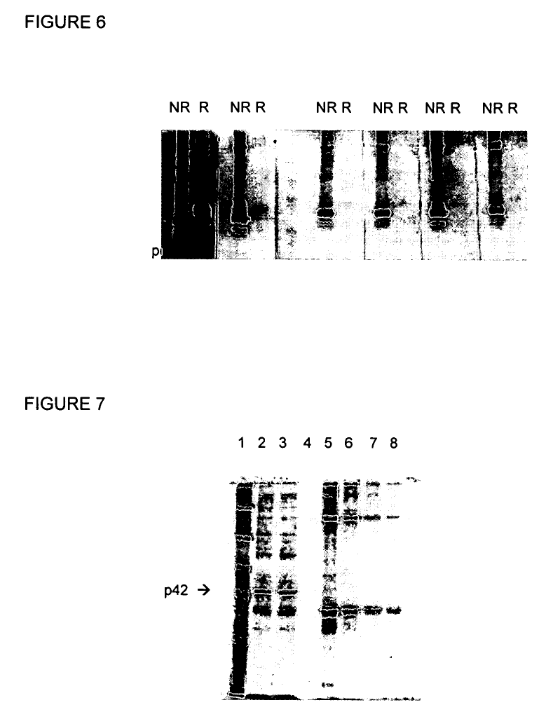

In contrast to other heterologous expression systems that have been used to express MSP-1 C-terminal subunits for use in malaria vaccine formulations, the Drosophila system provides a stable and continuous insect cell culture system that has the potential to produce large quantities of native-like subunits that maintain relevant immunological properties. As an example, the MSP-1 p42 protein expressed was determined to be reactive with a panel of conformationally sensitive monoclonal antibodies (see FIG. 6). The monoclonal antibodies 2.2, 7.5, 12.8 and 12.10 have been shown to bind to important epitopes on the parasite and in in vitro assays (Blackman et al 1990). The monoclonal antibody 5.2. (Chang et al, 1992) has been also shown to bind native MSP-1. The binding of these antibodies to non-reduced samples and their failure to bind to reduced samples indicates that they recognize conformationally dependent epitopes that are disulfide linked.

The MSP-1 C-terminal subunit proteins that are expressed and secreted from selected S2 cell lines as described and utilized in the preferred vaccine formulation are first purified by immunoaffinity methods. The anti-MSP-1 monoclonal antibody 5.2 (Chang et al, 1992) (“5.2 antibody” or “MAb 5.2”) is utilized for the purification. The 5.2 antibody is chemically conjugated to the appropriate column matrix by standard methods recommended by the manufacturer (NHS-Sepharose, Pharmacia, Piscataway, N.J.) to prepare suitable columns.

In a preferred embodiment, a vaccine formulation that combines (i) the Drosophila expressed MSP-1 C-terminal subunits as described herein with (ii) a water in oil emulsion adjuvant, preferably ISA51 (Aucouturier, J et al., Expert Rev. Vaccines (2002) 1:111-118) and (iii) a monophosphoryl lipid A derivative, preferably RC529 (Ulrich, J T and Myers K R, in Vaccine Design: The Subunit and Adjuvant Approach, ed. Powell, M F and Newman, M J (1995) Plenum Press, NY; Evans, J T et al., Expert Rev. Vaccines (2003) 2:219-229) potentiates a strong immune response. The use of such a vaccine formulation induces high titer parasite growth inhibiting antibodies in rabbits. The specificity of such a vaccine formulation to elicit parasite growth inhibiting antibodies is supported by the fact that the same recombinant antigens with other modern adjuvants failed to induce such a potent immune response. Furthermore, the vaccine formulation is capable of conferring protection from parasite challenge in the Aotus monkey model. Further details that describe the characteristics of the individual components and the efficacy of this vaccine formulation are contained below

The development of a vaccine formulation that has potential for human use is an important aspect in the development of a viable malaria vaccine. When adjuvants are utilized in a vaccine formulation, the use of adjuvants that are suitable for human use is critical. All of the adjuvants tested in combination with the MSP-1 C-terminal subunits in this work have been or have the potential to be used in humans. Specifically, the ISA51 and RC529 adjuvants are suitable for human use. ISA51 has been tested in several vaccine clinical trials involving over 1,000 patients (reviewed in Aucouturier, J et al., Expert Rev. Vaccines (2002) 1:111-118). ISA51 has been generally well-tolerated with only local and transient reactions reported. While RC529 has only been tested in a limited number of human subjects, it is a synthetic version with potentially improved safety profile of the monophosphoryl lipid A (MPL) adjuvant that has an extensive history of use in humans (reviewed in Evans, J T et al., Expert Rev. Vaccines (2003) 2:219-229).

Preferably, the MSP-1 C-terminal subunits are derived from the portion of the P. falciparum merozoite surface protein referred to as p42; in the FUP and 3D7 strains, p42 comprises amino acids Ala₁₃₃₃ to Ser₁₇₀₅ of MSP-1. The MSP-1 C-terminal subunit proteins, as described herein, are recombinantly produced and secreted from stably transformed insect cells. The MSP-1 C-terminal subunits may contain the entire p42 region of MSP-1 or portions thereof. More preferably, MSP-1 C-terminal subunits are derived from any of the three allelic types of P. falciparum; such as: K, MAD20, and Wellcome, as well as allelic types of P. vivax, P. malariae and P. ovale.

In all embodiments, the secretion of the MSP-1 C-terminal subunit proteins is typically directed by the tPA pre/pro secretion leader, but can be directed by any functional secretion signal capable of directing the expressed product through the insect cell secretion pathway and into the culture medium.

In a more preferred embodiment, the expressed MSP-1 C-terminal subunits are shown to maintain native-like characteristics of the C-terminal portion of the MSP-1 protein and are capable of eliciting a strong immune response when combined in a vaccine formulation with one or more appropriate adjuvants.

In an even more preferred embodiment, the immune response elicited is characterized by the presence of high levels of specific antibodies that are capable of inhibiting parasite growth in vitro.

In a further embodiment, the immune response elicited may contain enhancing antibodies (in addition to high levels of specific antibodies that are capable of inhibiting parasite growth). These enhancing antibodies, while not capable of inhibiting parasite growth in vitro, are characterized by their ability to enhance the parasite growth inhibition ability of the inhibitory antibodies.

While it is generally accepted that a strong adjuvant is needed to elicit an appropriate immune response to MSP-1 or subunits thereof, it is also important that the immune response also be specific in eliciting a high titer of parasite growth inhibiting antibodies. This may involve targeting specific antigen-presenting cell (APC) populations by means of specific adjuvant/antigen complexes that thereby result in more efficient uptake and antigen processing of the appropriate antigen epitopes. The optional addition of the RC529 adjuvant to the ISA51 adjuvant provides such a function.

The present invention thus concerns and provides a vaccine formulation as a means for preventing or attenuating infection by Plasmodium species. As used herein, a vaccine is said to prevent or attenuate disease if its administration to an individual results either in the total or partial immunity of the individual to the disease, i.e. a total or partial suppression of disease symptoms.

To immunize subjects against malaria, a vaccine formulation containing one or more subunits and one or more adjuvants is administered to the subject by means of conventional immunization protocols involving, usually, multiple administrations of the vaccine. The use of the immunogenic compositions of the invention in multiple administrations may result in the increase of antibody levels and in the diversity of the immunoglobulin repertoire expressed by the immunized subject.

Administration of the immunogenic composition is typically by injection, typically intramuscular or subcutaneous; however, other systemic modes of administration may also be employed.

According to the present invention, an “effective dose” of the immunogenic composition is one which is sufficient to achieve a desired biological effect. Generally, the dosage needed to provide an effective amount of the composition will vary depending upon such factors as the host's age, genetic background, condition, and sex. The immunogenic preparations of the invention can be administered by either single or multiple dosages of an effective amount. Effective amounts of the compositions of the invention can vary from 1-100 μg per dose, more preferably from 1-10 μg per dose.

The vaccines described here in can be used alone or in combination with other active malaria vaccines such as those containing other active subunits to the extent that they become available. These additional subunits can be from one or more of the developmental life cycle stages of the malaria parasite.

Although the descriptions presented above and the examples that follow are primarily directed at the expression of MSP-1 C-terminal subunits from Plasmodium falciparum, the methods and vaccine formulation can be applied to other Plasmodium species. For example P. vivax, P. malariae and P. ovale species also pose a health threat to humans. Formulations analogous to those for P. falciparum described herein can be developed as appropriate vaccines against P. vivax, P. malariae and P. ovale, respectively, for use in mammalian hosts.

### EXAMPLES

The examples below demonstrate the ability to effectively express the P. falciparum MSP-1 C-terminal subunit proteins from three strains of the parasite utilizing stably transformed insect cells. Examples are also given which demonstrate the expression of N-terminally truncated MSP-1 p42 subunit proteins. The purification of the expressed subunit proteins is demonstrated.

The examples further demonstrate that the ability of a particular adjuvant to enhance the immunogenicity of the MSP-1 C-terminal subunit vaccines is extremely variable and cannot be predicted a priori. The results presented below demonstrate that specific immune responses to the MSP-1 C-terminal subunit proteins formulated with different adjuvant combinations can vary from highly vigorous to undetectable. Thus, the selection of an effective immunogenic formulation must be determined empirically. Hence, the invention described herein is unique in its immunogenic properties.

Example 1 describes the construction of the expression plasmids for the MSP-1 C-terminal subunits. Examples 2 describes the construction of the N-terminally truncated MSP-1 p42 subunits as the experimental immunogen. Examples 3 to 7 and Examples 9 and 10 used purified MSP-1 p42 subunits as the experimental immunogen. Example 8 used purified MSP-1 N-terminally truncated p42 subunits as the experimental immunogen.

### Example 1

### Expression and Secretion of MSP-1 p42 Subunits in the Drosophila Expression System

A series of expression plasmids designed for the expression and selection of heterologous recombinant proteins in cultured Drosophila S2 cells was utilized for the work described. For details about the preparation of the expression plasmids, see U.S. Pat. Nos. 5,550,043; 5,681,713; 5,705,359; 6,046,025, the contents of which are fully incorporated herein by reference; in the event of conflict between those incorporated references and this instant, incorporating disclosure, the instant disclosure prevails. Specifically, the two plasmids utilized for this work are pMttbns and pCoHygro. The pMttbns expression vector contains the following elements: the Drosophila metallothioneine promoter (Mtn), the human tissue plasminogen activator (tPA) signal sequence, and the SV40 early polyadenylation signal (Culp et al, 1991). The pCoHygro plasmid provides a selectable marker for hygromycin (Van de Straten, 1989). The hygromycin gene is under the transcriptional control of the Drosophila COPIA transposable element long terminal repeat promoter. The pMttbns vector was modified by deleting a 15 base pair BamHI fragment which contained an extraneous Xho I site. This modified vector, referred to as pMttΔXho, allows for directional cloning of inserts utilizing unique Bgl II and Xho I sites. For details about the preparation of the expression plasmids and use in the Drosophila expression system, see commonly assigned U.S. Pat. Nos. 6,165,477, 6,416,763, 6,432,411, and 6,749,857, the contents of which are fully incorporated herein by reference; in the event of conflict between those incorporated references and this instant, incorporating disclosure, the instant disclosure prevails. Unless otherwise defined herein, the definitions of terms used in such commonly assigned patents shall apply herein. The DNA sequences cloned into the plasmids in such commonly assigned patents are, of course, different from, and superseded by, the cloned p42 sequences disclosed herein for the purposes of this document.

All constructs containing MSP-1 p42 sequences were made by cloning PCR amplified fragments into the expression vector pMttΔXho. Genomic DNA was prepared from cultured P. falciparum parasites of the three strains, FUP, NF54 (clone 3D7) and FVO utilizing the DNeasy Tissue Kit from Qiagen (Valencia, Calif.). The nucleotide sequences corresponding to the MSP-1 p42 subunits from FUP, 3D7, and FVO are shown in SEQ ID NOs:4, 5, and 6 respectively and the corresponding amino acid sequences encoded by these three nucleotide sequences are shown in SEQ ID NO:1, SEQ ID NO.2 and SEQ ID NO:3 respectively.

The MSP-1 fragments for the various MSP-1 p42 subunits were PCR amplified with oligonucleotide primers that were based on the published sequences of the three strains (FUP—Genbank accession number M37213, NF54 (clone 3D7) Genbank accession number Z35327, and FVO—Genbank accession number L20092). The amplified MSP-1 p42 PCR fragments contain the sequence encoding amino acids Ala₁₃₃₃ to Ser₁₇₀₅ of MSP-1 for the FUP strain, amino acids Ala₁₃₂₇ to Ser1699 of MSP-1 for the NF54 (clone 3D7) strain, and amino acids Ala₁ to Ser₃₅₅ of p42 for the FVO strain. In addition to the MSP-1 p42 specific sequences, the oligonucleotide primers encoded for appropriate restriction sites and stop codons. PCR amplification was accomplished by use of the high fidelity Pfx polymerase (Invitrogen, Carlsbad, Calif.). The resultant PCR amplified fragment was digested with appropriate restriction enzymes, Bgl II or Bam HI (compatible with Bgl II) and Xho I, and inserted into the pMttΔXho vector digested with Bgl II and Xho I. Cloning into the Bgl II site of pMttΔXho results in the addition of four amino acids, Gly-Ala-Arg-Ser, to the amino terminus of the protein expressed due to the fusion with the tPA leader sequence. The junctions and full inserts of all constructs were sequenced to verify that the various components that have been introduced are correct and that the proper reading frame has been maintained. The disclosures of the Drosophila expression system and corresponding purification system set forth in Ivy et al., U.S. Pat. No. 6,136,561; Ivy et al., U.S. Pat. No. 6,165,477; McDonnell et al., U.S. Pat. No. 6,416,763; Ivy et al., U.S. Pat. No. 6,432,411, are all fully incorporated herein by reference; in the event of conflict between those incorporated references and this instant, incorporating disclosure, the instant disclosure prevails (for instance, there are differences in the expression cassettes and in the immuno-affinity antibodies of the instant invention versus those used in the incorporated references).

Drosophila melanogaster S2 cells (“Drosophila S2 cells” or simply “S2 cells” Schneider, 1972) obtained from ATCC were utilized. Cells are adapted to growth in Excell 420 medium and all procedures and culturing are in this medium. Cells are passed between days 5 and 7 and are typically seeded at a density of 1×10⁶ cells/ml and incubated at 27° C. All expression plasmids containing the coding sequences for the MSP-1 p42 subunits from the three P. falciparum strains were transformed into S2 cells by means of the calcium phosphate method. The cells were co-transformed with the pCoHygro plasmids for selection with hygromycin B at a ratio of 20 μg of expression plasmid to 1 μg of pCoHygro. Following transformation, cells resistant to hygromycin, 0.3 mg/ml, were selected. Once stable cells lines were selected, they were evaluated for expression of the appropriate products. Five ml cultures were seeded at 2×10⁶ cells/ml and induced with 0.2 mM CuSO₄ and cultured at 27° C. for 7 days. Samples of culture medium were subjected to SDS-PAGE and Western blot analysis.

Immuno-affinity chromatography (IAC) methods have been utilized as a rapid means to purify expressed MSP-1 p42 subunit proteins for preliminary antigenic and immunogenic studies. The MSP-1 conformationally sensitive MAb 5.2 was used to prepare p42-specific IAC columns (Siddiqui et al 1987 and Chang et al 1992). Sufficient quantities of the MAb 5.2 were produced in a Cell Pharm 1000 hollow fiber bioreactor according to the manufacturer's recommendations (Unisyn, Hopkinton, Mass.). The MAb 5.2 hybridoma cell line was obtained from ATCC and grown in RPMI 1640 (Cambrex, Hopkins, Mass.). The medium was supplemented with FBS at 10% for growth in flask and 5% for growth in the hollow fiber bioreactor. The bioreactor was run for 25 days and resulted in a total yield of 57 mg. A two ml bed volume column was made by coupling 10 mg of affinity purified MAb 5.2 per ml of column matrix (activated N-hydroxy-succinimide-HiTrap, Pharmacia, Piscataway, N.J.). The IAC column was perfused with 200 ml of culture medium from a 400 ml spinner flask culture at a rate of 1 ml per minute. Following washing with 10 mM phosphate buffer, pH 7.2, the antigen was eluted with 100 mM glycine pH 2.5. The eluted product was neutralized with 1 M Tris, pH 7.5 (final concentration 0.2 M), and NaCl was added to a final concentration of 150 mM. The sample was then buffer exchanged into phosphate buffered saline (“PBS”) and concentrated by membrane ultrafiltration using a Centricon 30 (Millipore, Bedford, Mass.).

Examples of the expressed MSP-1 p42 subunit proteins in the culture medium from transformed S2 cells along with IAC purified MSP-1 p42 protein are shown in FIG. 6. The MSP-1 p42 subunits expressed in S2 cells have a molecular weight of approximately 42 kD. The concentration of the purified product was determined based on values obtained from total amino acid analysis. A comparison of dilutions of purified MSP-1 p42 subunit proetins to the material in culture medium results in an estimate of 30 μg/ml for the expression level of the transformants, as detected in the SDS-PAGE result shown in FIG. 7.

In the development of a malaria vaccine that includes MSP-1 C-terminal subunits, it is important that a native-like structure is maintained. The MSP-1 p42 subunit proteins expressed were determined to be reactive with a panel of conformationally sensitive monoclonal antibodies (see FIG. 6). The MAb 5.2. (Chang et al, 1992) has been shown to bind native MSP-1. MAbs 2.2, 7.5, 12.8 and 12.10 have been shown to bind to important epitopes on the parasite in vitro assays and are known to bind to conformationally sensitive epitopes (Blackman et al 1990). MAbs 2.2, 7.5, 12.8 and 12.10 were used only for comparison in the Western Blot in FIG. 6 and were not used in purification of the subunit proteins. The binding of these antibodies to non-reduced samples and their failure to bind to reduced samples indicates that they recognize conformationally dependent epitopes that are disulfide linked. These results indicate that the Drosophila expressed MSP-1 p42 subunit proteins maintained a native-like structure.

### Example 2

### Expression and Secretion of N-terminally truncated MSP-1 p42 Subunits in the Drosophila Expression System

There are many studies that have utilized either the p19 or p42 subunits of MSP-1 in an effort to develop a malaria vaccine. There have been reports of success for each of these MSP-1 subunits as well as failures with each of these subunits, as reviewed above and further discussed below. Because of the conflicting results, it is not clear which regions of these subunits are most relevant in regards to their ability to serve as antigens in eliciting antibody responses that are capable of inhibiting parasite growth in vitro or in the protection of animals to challenge. It has been hypothesized that the induction of growth-inhibitory antibodies to the p19 region is determined by the specificity of T helper epitopes in p33 region. In support of this theory, a study that directly compared the ability of recombinant p19 and p42 subunits to elicit parasite growth inhibitory antibodies was conducted (Hui et al, 1994). Using the same adjuvant (FCA), both p19 and p42 recombinants elicited high titers of antibodies based on ELISA determination. However, the antibodies generated by the p42 antigen were inhibitory against parasite growth were as the antibodies generated by the p19 were incapable of inhibiting parasite growth. Despite the inability of the p19 generated antibodies to inhibit parasite growth, it was demonstrate that of the p42 generated antibodies capable of binding to parasite-derived MSP-1 could be cross-absorbed using the p19 antigen. Also in support of this theory, a study by Udhayakumar et al (1995) characterized B and T cell epitopes in the p42 region relative to the immune response of individuals living in Kenya, a region in which falciparum malaria is highly endemic. This study revealed that B cell epitopes were mainly in the p19 region and that T cell epitopes capable of providing proliferative responses were located in the p33 region. While these T cell epitopes were not determined to provide T helper function, it was suggested that they may be responsible for focusing the antibody response to the important parasite growth inhibiting epitopes of p19. While these results support the theory of T-cell helper functions in the p33 region, there is no data on the expression of recombinant subunits that are designed to enhance the ability to elicit parasite growth inhibitory antibodies or protective responses in animal models.

A series of expression plasmids were designed for the expression of MSP-1 p42 variants in which segments of the N-terminal portion of protein were removed in an effort to define regions of p33 that could enhance the ability to elicit parasite growth inhibitory antibodies or protective responses in animal models. Computer programs were used to aid in the analysis to guide the selection of appropriate segments of p33 to retain relevant T-cell epitopes. First, the p33 region was analyzed for the presence of sequences that fit the pattern established for T-cell epitopes (Margalit et al, 1987). The algorithm is part of a computer program written by Menendez-Arias and Rodriguez (1990) for selecting potential T-cell epitopes. This analysis resulted in the prediction of 14 T-cell epitopes. The epitope with the highest amphipathic score is one that is in the conserved region at the N-terminus, LKPLAGVYRSLKKQ. This epitope is referred to as CT (conserved T). The next epitope that was identified is positioned 72 amino acids preceding the start of the p19 sequence. The peptide, AHVKITKLSDLKAID, is referred to as C72. Based on the identification of these two epitopes, two subunits that have a large portion of the p33 region removed were designed. These two recombinants are referred to as C72p19 and CTC72p19. The “72” of C72 refers to the number of p33 C-terminal amino acid residues that precede p19 . The CTC72 subunit additionally contains the conserved N-terminal T-cell epitope (CT) fused to the N-terminus of C72p19. The two C72 containing subunits are based on the 3D7 sequence. The nucleotide sequences for the C72p19 and CTC72p19 N-terminally truncated MSP-1 p42 subunits are shown in SEQ ID NO:11 and SEQ ID NO:12 respectively and the corresponding amino acid sequences encoded by these two nucleotide sequences are shown in SEQ ID NO:8 and SEQ ID NO:9 respectively. In FIG. 5 an alignment of the the amino acid sequences with the p42 sequence is shown.

A third subunit, C31p19 was also constructed. This subunit was based on further analysis of MHC class II epitopes in the p33 region with the computer program TEPITOPE (Sturniolo T. et al, Nat. Biotechnology (1999) 17:555-561; Singh, H. and Raghava, G. P. S. (2001) Bioinformatics, 17(12), 1236-37) and on experimental data gained with peptides designed from the results of this program. The C31p19 subunit is based on the FUP sequence. The nucleotide sequence for the C31p19 N-terminally truncated MSP-1 p42 subunit is shown in SEQ ID NO:10 and the corresponding amino acid sequence encoded by this nucleotide sequence is shown in SEQ ID NO:7. The amino acid alignment of C31p19 is also shown in FIG. 5 relative to p42.

The Drosophila expression plasmids utilized were the same as described in Example 1. Expression constructs were made by directly cloning PCR amplified fragments, or subcloning fragments from shuttle vectors containing fragments that were chemically synthesized, into the expression vector pMttΔXho. Genomic DNA that was used for template for PCR amplification of MSP-1 fragments was prepared from cultured P. falciparum parasites of the strain NF54 (clone 3D7) utilizing the DNeasy Tissue Kit from Qiagen.

Oligonucleotide primers were designed based on the published sequence for the NF54 strain (Genbank accession number Z35327). In addition to the MSP-1 specific sequences, the oligonucleotide primers encoded for appropriate restriction sites and stop codons. The PCR amplification, cloning, and transformation of S2 cells were accomplished as described in Example 1. The expressed subunit proteins were purified by IAC methods as described in Example 1.

### Example 3

### Evaluation of the Immunogenicity of Drosophila Expressed MSP-1 p42 Subunit Proteins in Rabbits

The immunogenicity of the Drosophila expressed FUP MSP-1 p42 subunit protein formulated with either Freund's complete adjuvant (FCA) or Iscomatrix (CSL, Victoria, Australia) was evaluated in rabbits. These formulations are referred to as FCA/p42 and Isco/p42, respectively. New Zealand White rabbits were immunized with four intramuscular doses of purified FUP MSP-1 p42 protein, 50 μg/dose, once every 4 weeks. Rabbits were bled two weeks after the first three doses and at three and four weeks after the fourth dose. The sera were then assessed for anti-p42 titers by ELISA. The sera were also tested for the ability to inhibit parasite growth in vitro (Hui and Siddiqui, 1987). Briefly, to assay for parasite growth inhibition, the serum sample is added to culture medium to give a final concentration of 30% and incubated with infected human erythrocytes that are adjusted to an initial parasitemia of 0.5%. P. falciparum 3D7 parasites that are adapted to growth in human serum are utilized in the assay. Cultures are then incubated for 72 hours, and the parasitemia of Giemsa-stained thin smears of the cultured erythrocytes are determined by microscopy. The percent inhibition is calculated by subtracting the parasitemia in test samples from the parasitemia in control serum samples (pre-bleeds from the same animal) and dividing by the parasitemia in control serum sample and then multiplying by 100.

The ELISA titers and the parasite inhibition results are presented in Table 1. The serum from the third and fourth bleeds was tested for parasite growth inhibition activity. Only sera from the FCA/p42 immunized rabbits resulted in the inhibition of parasite growth. The lowest ELISA titer corresponds to the least parasite growth inhibition for the FCA/p42 immunized rabbits. The results from the FCA/p42 immunized rabbits demonstrate that the FCA/p42 vaccine formulation has appropriate immunological characteristics. The results from the Iscomatrix immunized rabbits demonstrate that high antibody titers (for example, rabbit 7863) do not always result in the ability to inhibit parasite growth. These results support the concept that it is necessary for the immune response to be potentiated in an appropriate manner in order to achieve a response with the appropriate functional activity. Since the same antigen preparation was used, it appears that the adjuvant used has an important effect on the nature and specificity of the response and demonstrates that adjuvant selection plays a critical role in the ability of MSP-1 p42 subunit protein to elicit parasite inhibitory antibodies. Furthermore, these results also demonstrate that the immunization of rabbits and the testing of the resultant serum in the in vitro parasite growth inhibition assays provides a means of evaluating the ability of a given antigen/adjuvant combination to elicit an appropriate immune response with MSP-1 p42 subunit proteins.

TABLE 1
ELISA titration and inhibition results on serum from rabbits immunized with
MSP-1 p42

| | | ELISA Titer* | ELISA Titer* after | | ELISA Titer* after | |
|---|---|---|---|---|---|---|
| | | after 2nd | 3rd immunization | | 4th immunization | |
| | | immunization and | and O.D. (in | % inhibition after | and O.D. (in | % inhibition after |
| Adjuvant | Rabbit ID | O.D. (in paren.) | paren.) | 3rd immunization | paren.) | 4th immunization |
| Iscomatrix | 7863 | 625 k (0.116) | 625 K (0.161) | 0 | 625 K (0.107) | 0 |
| | 7864 | 125 k (0.362) | 125 K (0.295) | 0 | 125 K (0.224) | 0 |
| | 7865 | 125 k (0.265) | 125 K (0.303) | 0 | 125 K (0.201) | 0 |
| Freund's | 7866 | 3,125 k (0.124) | 3,125 K (0.113) | 80 | 3,125 K (0.322) | 84 |
| | 7867 | 625 k (0.220) | 625 K (0.287) | 57 | 625 K (0.277) | 60 |
| | 7868 | 3,125 k (0.107) | 3,125 K (0.154) | 85 | 3,125 K (0.119) | 87 |

*dilution (k = ×1000) which gives an O.D. (Optical Density) of 0.1 above background
(O.D. values reported are after subtraction of background)

The results from antigenic and immunogenic analysis of the Drosophila expressed MSP-1 p42 subunit, Examples 1 and 3 respectively, demonstrate that this recombinantly expressed subunit protein has appropriate antigenic and immunogenic properties and is therefore a viable malaria vaccine candidate.

### Example 4

### MSP-1 p42 Subunits Combined with Various Adjuvants Engender Varying Immune Responses in Mice

Groups of Balb/c mice were immunized with four subcutaneous doses of purified FUP MSP-1 p42 protein, 10 μg/dose, once every 4 weeks, using the adjuvants Montanide ISA720, Montanide ISA51, QS21, and RC529, either individually or in combination as shown in Table 2. A first portion of mice from each group were sacrificed 7 days after the final dose and their spleens were removed. Splenocytes were cultured and used for proliferation and cytokine analysis. The remaining mice from each group were exsanguinated 14 days after the final dose and sera was collected. Sera from mice within each group were pooled. The sera were then assessed for anti-p42 titers by ELISA and also for IgG subtype. The results for the various analyses are shown in Table 2 below.

TABLE 2
Immunological responses of Balb/c mice immunized with FUP MSP-1 p42 and
a variety of adjuvants.

| | | | ELISA | Proliferation | IFN | | | |
|---|---|---|---|---|---|---|---|---|
| Group | Adjuvant | Antigen | Titer* | SI | Gamma+ | IL4+ | IL5+ | IL10+ |
| 1 | Freund's | FUP p42 | 1/64,000 | 15.7 | 0.9 | 0.2 | 0.2 | 0.3 |
| | | 10 μg | | | | | | |
| 2 | RC529/QS21 | FUP p42 | 1/256,000 | 43.7 | 6.5 | 3.4 | 2.2 | 3.3 |
| | | 10 μg | | | | | | |
| 3 | ISA 720 | FUP p42 | 1/256,000 | 4.7 | 5.9 | 3.0 | 4.3 | 4.2 |
| | | 10 μg | | | | | | |
| 4 | ISA 720/RC529 | FUP p42 | 1/256,000 | 119.6 | 1.9 | 0.4 | 0.1 | 0.5 |
| | | 10 μg | | | | | | |
| 5 | ISA 720/QS21 | FUP p42 | 1/256,000 | 82.6 | 2.3 | 1.4 | 2.5 | 1.4 |
| | | 10 μg | | | | | | |
| 6 | ISA 720/RC529/ | FUP p42 | 1/256,000 | 51.8 | 4.0 | 0.3 | 0.3 | 0.5 |
| | QS21 | 10 μg | | | | | | |
| 7 | ISA 51 | FUP p42 | 1/256,000 | 97.4 | 6.6 | 5.4 | 4.4 | 3.7 |
| | | 10 μg | | | | | | |
| 8 | ISA 51/RC529 | FUP p42 | 1/256,000 | 98.5 | 4.5 | 2.9 | 4.1 | 4.1 |
| | | 10 μg | | | | | | |
| 9 | ISA 51/QS21 | FUP p42 | 1/256,000 | 116.8 | 5.4 | 3.8 | 3.2 | 3.0 |
| | | 10 μg | | | | | | |
| 10 | ISA 720/RC529/ | No | 0 | 4.5 | 0.1 | 0.0 | 0.0 | 0.0 |
| | QS21 | antigen | | | | | | |

*dilution that results in an OD 3 times greater than background (0.07)
+ng/ml in bulk culture 7 days post immunization as determined by quantitative ELISA

Groups of Swiss Webster mice were immunized with four subcutaneous doses of purified FUP MSP-1 p42 subunit, 10 μg/dose, once every 4 weeks with individual or combinations of adjuvants as shown in Table 3. A portion of mice from each group were sacrificed 7 days after the final dose and their spleens were removed. Splenocytes were cultured and used for proliferation and cytokine analysis. The remaining mice from each group were exsanguinated 14 days after the final dose and sera was collected. Sera from mice within each group were pooled. The sera were then assessed for anti-p42 titers by ELISA and also for IgG subtype. The results for the various analyses are shown in Table 3 below.

TABLE 3
Immunological responses of Swiss Webster mice immunized with FUP MSP-1
p42 and a variety of adjuvants.

| | | | ELISA | Proliferation | IFN | | | |
|---|---|---|---|---|---|---|---|---|
| Group | Adjuvant | Antigen | Titer* | SI | Gamma+ | IL4+ | IL5+ | IL10+ |
| 1 | RC529 | FUP p42 | 1/256,000 | 7.3 | 0.8 | 0.0 | 0.3 | 0.3 |
| | | 10 μg | | | | | | |
| 2 | QS21 | FUP p42 | 1/1,024,000 | 1.7 | 5.6 | 0.3 | 2.8 | 5.6 |
| | | 10 μg | | | | | | |
| 3 | RC529/QS21 | FUP p42 | 1/1,024,000 | 43.6 | 21.5 | 0.0 | 1.8 | 2.9 |
| | | 10 μg | | | | | | |
| 4 | ISA 720 | FUP p42 | 1/1,024,000 | 0.8 | 3.8 | 0.1 | 2.7 | 3.0 |
| | | 10 μg | | | | | | |
| 5 | ISA 720/RC529 | FUP p42 | 1/1,024,000 | 2.2 | 2.3 | 0.0 | 1.4 | 1.8 |
| | | 10 μg | | | | | | |
| 6 | ISA 720/QS21 | FUP p42 | 1/1,024,000 | 3.4 | 5.7 | 0.1 | 2.1 | 3.2 |
| | | 10 μg | | | | | | |
| 7 | ISA 51 | FUP p42 | 1/256,000 | 8.3 | 1.9 | 0.0 | 1.5 | 1.8 |
| | | 10 μg | | | | | | |
| 8 | ISA 51/RC529 | FUP p42 | 1/1,024,000 | 3.9 | 7.3 | 0.0 | 1.3 | 1.4 |
| | | 10 μg | | | | | | |
| 9 | ISA 51/QS21 | FUP p42 | 1/1,024,000 | 4.3 | 19.1 | 0.0 | 3.0 | 2.3 |
| | | 10 μg | | | | | | |
| 10 | ISA 51/QS21 | 3D7 p42 | 1/256,000 | 31.6 | 11.0 | 0.0 | 3.0 | 2.3 |
| | | 10 μg | | | | | | |
| 11 | ISA 51/QS21 | No | 0 | 2.0 | 0.7 | 0.0 | 0.0 | 0.0 |
| | | antigen | | | | | | |

*dilution that results in an OD 3 times greater than background (0.07)
+ng/ml in bulk culture 7 days post immunization as determined by quantitative ELISA

### Example 5

### MSP-1 p42 Subunits Combined with Various Adjuvants Engender Varying Immune Responses in Rabbits

Groups of New Zealand White rabbits were immunized with four intramuscular doses of purified FUP MSP-1 p42 protein, 50 μg/dose, once every 4 weeks using individual or combinations of adjuvants as shown in Table 4. Rabbits were bled two weeks after the first three doses and at three and four weeks after the fourth dose. The sera were then assessed for anti-p42 titers by ELISA. The sera were also tested for the ability to inhibit parasite growth in vitro (Hui and Siddiqui, 1987) as described in Example 3. Serum samples were tested for the presence of antibodies capable of parasite growth inhibition following the third and fourth doses. The results of the inhibition along with serum titers are presented in Table 4. The results shown in Table 4 above demonstrate that the ability of a particular adjuvant to enhance the immunogenicity of the FUP MSP-1 p42 subunit is extremely variable and is difficult to predict accurately a priori. Thus, the selection of an effective vaccine formulation is unexpected, and must be determined empirically. The adjuvants ISA 51 alone and ISA51 in combination were selected for further evaluation.

TABLE 4
ELISA values and parasite growth inhibition values for rabbits immunized with
MSP-1 p42

| | | | | Rabbit | ELISA | Percent |
|---|---|---|---|---|---|---|
| Group | Adjuvant | Antigen and dose | Route | ID | Titer* | Inhibition |
| 1 | Freund's | FUP | IM | 1 | 1/1,024,.000 | 33.8 |
| | | 50 μg | | 2 | 1/1,024,.000 | 74.0 |
| | | | | 3 | 1/1,024,.000 | 102.6 |
| 2 | ISA 720 | FUP | IM | 4 | 1/256,000 | No data |
| | | 50 μg | | 5 | 1/256,000 | −7.2 |
| | | | | 6 | 1/1,024,.000 | 76.1 |
| 3 | ISA 720/RC529 | FUP | IM | 7 | 1/16,000 | −25.7 |
| | | 50 μg | | 8 | 1/1,024,.000 | 99.3 |
| | | | | 9 | 1/64,.000 | −45.2 |
| 4 | ISA 720/QS21 | FUP | IM | 10 | 1/256,000 | 22.6 |
| | | 50 μg | | 11 | 1/1,024,.000 | 23.8 |
| | | | | 12 | 1/256,000 | 76.1 |
| 5 | ISA 720/QS21/RC529 | FUP | IM | 13 | 1/1,024,.000 | 35.2 |
| | | 50 μg | | 14 | 1/256,000 | 80.0 |
| | | | | 15 | 1/64,000 | 50.3 |
| 6 | ISA 51 | FUP | IM | 16 | 1/1,024,.000 | 95.5 |
| | | 50 μg | | 17 | 1/256,000 | 54.4 |
| | | | | 18 | 1/256,000 | 16.7 |
| 7 | ISA 51/RC529 | FUP | IM | 19 | 1/256,000 | 22.8 |
| | | 50 μg | | 20 | 1/256,000 | 104.1 |
| | | | | 21 | 1/256,000 | 56.1 |
| 8 | ISA 51/QS21 | FUP | IM | 22 | 1/64,000 | No data |
| | | 50 μg | | 23 | 1/16,000 | −74.6 |
| | | | | 24 | 1/64,000 | 17.8 |
| 9 | ISA 51/QS21/RC529 | FUP | IM | 25 | 1/64,000 | −7.1 |
| | | 50 μg | | 26 | 1/64,000 | No data |
| | | | | 27 | 1/256,000 | 76.9 |
| 10 | ISA 51/QS21/RC529 | No antigen | IM | 28 | — | 18.3 |
| | | | | 29 | — | −1.0 |
| | | | | 30 | — | 0.0 |

*dilution that results in an OD 3 time greater than background (0.09)

### Example 6

### MSP-1 p42 Subunit Formulated with ISA 51 alone or ISA 51 Combined with RC529 Elicits High Levels of Antibodies Capable of Inhibiting Parasite Growth

Groups of New Zealand White rabbits were immunized with four intramuscular doses of purified FUP, 3D7, or FVO MSP-1 p42 subunit, 50 μg/dose, once every 4 weeks using the following adjuvants, Freund's, ISA 51 alone, or ISA 51 in combination with RC529, as shown in Table 5. Rabbits were bled two weeks after the first three doses and at three and four weeks after the fourth dose. The sera were then assessed for anti-p42 titers by ELISA. The sera were also tested for the ability to inhibit parasite growth in vitro (Hui and Siddiqui, 1987) as described in Example 3. Serum samples were tested for parasite growth inhibition following the third and fourth doses. The results of the inhibition along with serum titers from the four weeks post fourth dose serum samples are presented in Table 5.

TABLE 5
Immunization of Rabbits with MSP-1 p42 subunit and ISA 51 alone and ISA 51
in combination with RC529.

| | | | | Rabbit | ELISA | Percent |
|---|---|---|---|---|---|---|
| Group | Adjuvant | Antigen and dose | Route | Number | Titer* | Inhibition |
| 1 | Freund's | FUP | IM | 1 | 1/1,024,000 | 108.2 |
| | | 50 μg | | 2 | 1/1,024,000 | 104.8 |
| | | | | 3 | 1/256,000 | 57.6 |
| 2 | ISA 51 | FUP | IM | 4 | 1/1,024,000 | 70.4 |
| | | 50 μg | | 5 | 1/256,000 | 90.4 |
| | | | | 6 | 1/1,024,000 | 111.6 |
| 3 | ISA 51/RC529 | FUP | IM | 7 | 1/256,000 | 79.4 |
| | | 50 μg | | 8 | 1/1,024,000 | 84.9 |
| | | | | 9 | 1/4,096,000 | 109.0 |
| 4 | ISA 51/RC529 | FVO | IM | 10 | 1/1,024,000 | 45.2 |
| | | 50 μg | | 11 | 1/1,024,000 | 77.9 |
| | | | | 12 | 1/1,024,000 | 89.1 |
| 5 | ISA 51/RC529 | 3D7 | IM | 13 | 1/1,024,000 | 80.6 |
| | | 50 μg | | 14 | 1/256,000 | 31.8 |
| | | | | 15 | 1/1,024,000 | 98.0 |
| 6 | ISA 51/RC529 | No antigen | IM | 16 | — | −4.6 |
| | | 50 μg | | 17 | — | −54.7 |
| | | | | 18 | — | −34.5 |

*dilution that results in an OD 3 time greater than background (0.09)

The results given in Table 5 above demonstrate that the adjuvants ISA51 alone and ISA 51 in combination with RC529 provide strong (greater than 50% inhibition) responses in all three rabbits in the groups immunized with the FUP MSP-1 p42 subunit, which is similar to the response elicited in rabbits immunized with Freund's adjuvant (group 1). The rabbits immunized with the 3D7 and FVO MSP-1 p42 subunit mixed with ISA51 and RC529 resulted in 2 of the 3 rabbits having strong responses. Therefore, these adjuvants which have potential to be used in humans elicit immune responses that are on a level similar to those elicited by Freund's Adjuvant (‘the gold standard”), which is not suitable for use in humans.

### Example 7

### Evaluation of the Protective Efficacy Elicited by MSP-1 p42 Subunits Formulated with ISA 51 Alone and the Combination of ISA 51 and RC529 in the Aotus Nancymai Challenge Model

The Aotus nancymai monkey trial utilized 18 monkeys. They were randomly assigned into three groups of six each. Group one consisted of control animals that were immunized with adjuvant only. Animals in group two were immunized with 50 μg of FUP MSP-1 p42 subunit formulated with Montanide ISA51. Animals in group three were immunized with 50 μg of FUP MSP-1 p42 subunit formulated with the combination of Montanide ISA51 and RC529. A total of four immunizations were given at 0, 1, 3, and 6 months. Each dose was administered intramuscularly. Serum was collected from the monkeys every two weeks during the trial. Fourteen days after the last dose the monkeys were challenged with 50,000 FUP infected erythrocytes. Blood samples were taken for 54 days following challenge and the number of parasite infected red blood cells was determined.

Serum samples were assessed for anti-p42 titers by ELSIA and IFA. Sera were also assessed for the presence of parasite growth inhibition antibodies as described in Example 3. Only serum samples taken after the third and fourth doses were tested for parasite growth inhibition. The results of these analyses are summarized in Table 6.

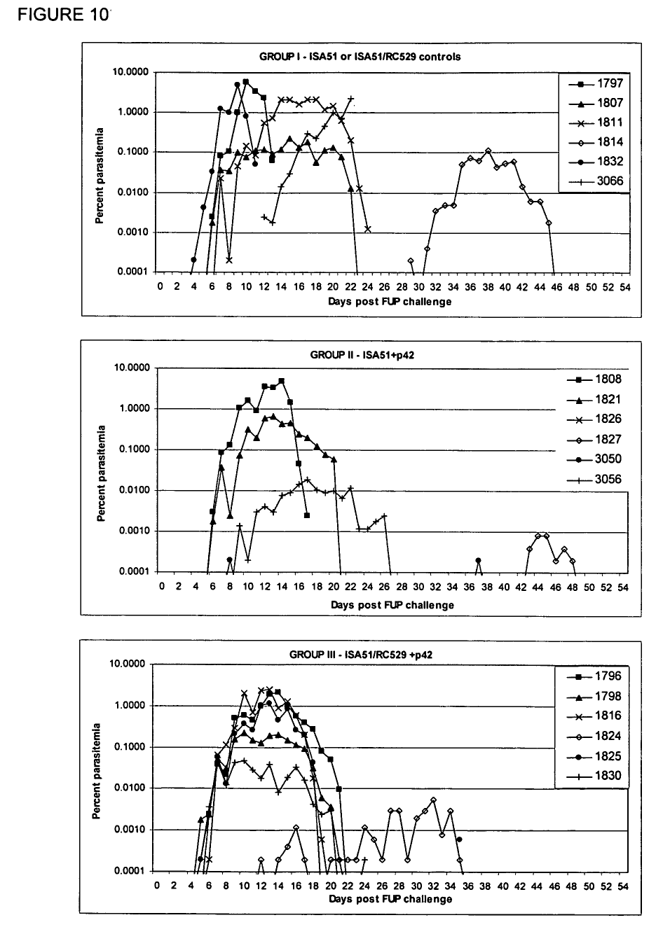

The course of parasitemia in the monkeys post challenge in presented in FIG. 10. Four of 6 control monkeys in Group 1 had peak parasitemias>2% of which two required drug treatment. The other 2 monkeys had peak parasitemias<2% and self cured. In Group 2 only 1 monkey required drug treatment and the other 5 controlled parasite growth. Three of these 5 animals had no or extremely low parasite counts and the other 2 had peak parasitemias <0.5%. In Group 3, all monkeys were able to control parasite growth but to various degrees. Three monkeys had low peak parasitemias (<0.23%), while that of the other three animals were between 1.2% and 2.5%. The parasitemia data following challenge is summarized in Table 7.

TABLE 6
Analyses of serum samples taken from Aotus monkeys.

| | | ½ max | IFA titer | | % inhibition |
|---|---|---|---|---|---|
| | Monkey | ELISA titer | post 4th | % inhibition | post |
| | # | post 4th dose | dose | post 3rd dose | 4th dose |
| Group 1 | 1797 | ** | <32 | 50.0 | −48.2 |
| | 1807 | ** | 32 | ND | −6.2 |
| | 1811 | ** | <32 | −9.3 | −26.6 |
| | 1814 | 4410 | <32 | 0.0 | 1.5 |
| | 1832 | 87 | <32 | 35.5 | 1.1 |
| | 3066 | 71 | 32 | 52.5 | −3.5 |
| Group 2 | 1808 | 99554 | 8192 | 45.2 | 30.5 |
| | 1821 | 105222 | 16384 | 52.6 | −19.4 |
| | 1826 | 99194 | 8192 | 18.5 | −3.9 |
| | 1827 | 889803 | >65536 | 104.1 | 100.0 |
| | 3050 | 570498 | 32768 | 98.2 | 94.9 |
| | 3056 | 153206 | 16384 | 90.4 | −28.1 |
| Group 3 | 1796 | 162856 | 8192 | 31.4 | −35.3 |
| | 1798 | 242394 | 32768 | ND | 48.8 |
| | 1816 | 119405 | >65536 | 48.9 | −70.0 |
| | 1824 | 784647 | 8192 | 82.1 | 77.6 |
| | 1825 | 47607 | 4096 | 48.0 | 6.0 |
| | 1830 | 428741 | 32768 | 43.1 | 19.8 |

TABLE 7
Summary of data following the challenge of the Aotus monkeys

| | | | Average | Peak | Mean+ | Cumulative | Mean+ |
|---|---|---|---|---|---|---|---|
| | | Day of | Day of | Parasitemia | Peak | Parasitemia | Cumulative |
| | Monkey | Patentcy | Patentcy | (parasites/μl) | Parasitemia | Counts | Parasitemia |
| Group 1 | 1814* | 8** | 7.2 | 5,760 | 62,449 | 25,231 | 205,986 |
| | 1807 | 6 | | 11,340 | | 81,772 | |
| | 3066 | 12 | | 113,220 | | 256,500 | |
| | 1832 | 4 | | 248,000 | | 362,761 | |
| | 1797 | 6 | | 296,000 | | 527,881 | |
| | 1811 | 7 | | 109,260 | | 753,786 | |
| Group 2 | 1826* | none | 8.5 | 0 | 379 | 0^{§} | 1,138 |
| | 3050* | 8 | | 10 | | 20^{§} | |
| | 1827* | 22** | | 40 | | 141^{§} | |
| | 3056 | 9 | | 930 | | 5,812^{§} | |
| | 1821 | 6 | | 32,760 | | 172,562 | |
| | 1808 | 6 | | 244,000 | | 769,731 | |
| Group 3 | 1824 | 12 | 6.5 | 270 | 13,330 | 1,285^{§} | 67,974 |
| | 1830 | 5 | | 2,340 | | 16,084 | |
| | 1798 | 5 | | 11,430 | | 77,372 | |
| | 1825 | 5 | | 58,000 | | 246,842 | |
| | 1796 | 6 | | 108,000 | | 454,602 | |
| | 1816 | 6 | | 124,000 | | 549,702 | |

*2^{nd} FUP challenge on day 21
**based on 2^{nd} challenge
+geometric mean
^{§}protected based on CPC < 10,000

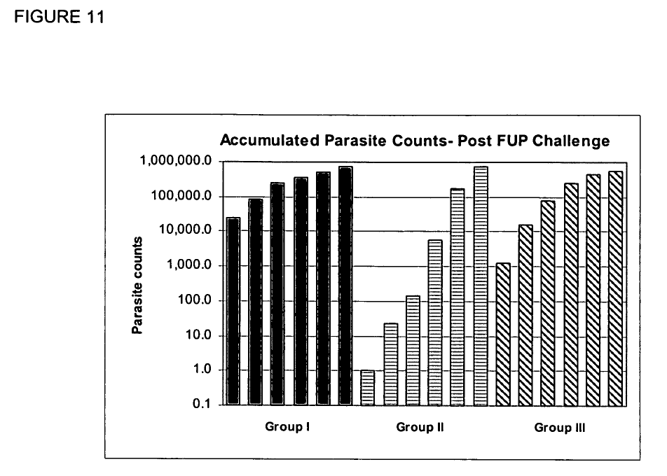

The cumulated parasite counts (“CPC”) were used as primary endpoint to assess protection. The CPC values for each group in ranked order are presented in FIG. 11. Based on the CPC values it was determined that 4 monkeys in Group 2 and 1 monkey in Group 3 were protected (CPC<10,000/μl). These results are significant in that this level of protection was achieved with an adjuvant other than Freund's Adjuvant and this adjuvant has the potential for human use.

### Example 8

### Evaluation of the Immunogenicity of N-terminally Truncated MSP-1 p42 Subunits in Rabbits

Groups of New Zealand White rabbits were immunized with four intramuscular doses of the purified N-terminally truncated MSP-1 p42 subunits C72p19 and CTC72p19. Each dose contained 50 μg of subunit protein. A total of 4 doses were administered once every 4 weeks. All doses were mixed with the adjuvant combination of ISA 51 and RC529. Rabbits were bled two weeks after the first three doses and at three and four weeks after the fourth dose. The sera were then assessed for anti-p42 titers by ELISA. Sera were also assessed for the presence of parasite growth inhibition antibodies as described in Example 3. Only serum samples taken after the third and fourth doses were tested for parasite growth inhibition. The results of the inhibition, along with serum titers from the third and fourth dose (4 weeks post immunization) serum samples, are presented in Table 8.

TABLE 8
Immunization of Rabbits with N-terminally truncated MSP-1 p42 subunits
C72p19 and CTC72p19 with ISA 51 in combination with RC529.

| | | | ELISA | % | ELISA |
|---|---|---|---|---|---|
| | | % inhibition | OD | inhibition | OD |
| | Rabbit | after the | after the | after the | after the |
| Antigen | Number | 3^{rd} dose^{a} | 3^{rd} dose^{a,b} | 4^{th} dose^{b,c} | 4^{th} dose^{b,c} |
| C72p19 | 19 | 53.6 | 0.70 | 75.5 | 0.69 |
| | 20 | 14.1 | 0.25 | 37.5 | 0.43 |
| | 21^{d} | X | X | X | X |
| CTC72p19 | 22 | 0 | 0.19 | 0 | 0.19 |
| | 23 | 69.1 | 0.88 | 66.7 | 0.73 |
| | 24 | 32.7 | 0.37 | 23.3 | 0.54 |

^{a}21 days post immunization
^{b}OD at a serum dilution of 1/256,000
^{c}28 days post immunization
^{d}rabbit died

The results presented in Table 8 above demonstrate that the two N-terminally truncated MSP-1 p42 subunits are capable of eliciting antibodies that are capable of inhibiting greater than 50% of the parasites in the in vitro assay in some the rabbits immunized. These results indicate that while these subunits are still capable of eliciting parasite growth inhibition antibodies, the strength of the responses are not as strong as those elicited with the full p42 subunit.

### Example 9

### Identification of MHC Class II T-cell Epitopes in MSP-1 p42 C-terminal Subunits

The immunization of mice with C72p19 and CTC72p19 subunits demonstrates that these subunits still are capable of eliciting strong, general antibody responses (ELISA titers) directed at the C-terminal region of MSP-1 (data not shown). However, the goal of reliably enhancing the production of specific parasite growth inhibitory antibody does not appear to have been achieved as determined by the results from rabbits presented in Example 8. While a greater potential for eliciting parasite growth inhibitory antibodies is not apparent from the use of the N-terminally truncated MSP-1 p42 subunits, the deletion of a large portion of the p33 region did not entirely eliminate the ability to produce inhibitory antibodies (1 rabbit from each group resulted in strong inhibitory antibodies, >50%).

As discussed earlier, it has been hypothesized that the p33 region of p42 provides T-cell help in directing antibody responses against the p19 region. Based on the results presented, it is not yet clear whether these segments provide T-cell help. In an effort to further characterize the p33 region for T-cell help epitopes, the p33 region was evaluated for MHC class II epitopes. Specifically, the region was assessed for the presence of peptides with binding specificity for the HLA-DR isotype which is the predominant MHC II isotype. This was accomplished through the use of the TEPITOPE software developed to predict promiscuous HLA ligands (Sturniolo, T. et al, Nat. Biotechnology (1999) 17:555-561; Singh, H. and Raghava, G. P. S. (2001) Bioinformatics 17(12), 1236-37). Based on analysis of the p33 region with the TEPITOPE program (threshold value of 3% utilized), eight peptides were selected (see Table 9). The original CT peptide was also identified by the program; however, with this analysis, the ends of the peptide were modified to extend the peptide to fit the predicted epitope. The new peptide, VIYLKPLAGVYRSLKKQIE is referred to as CT-2. The C72 peptide that is described in Example 2 and tested in mice in Example 9 was not identified by the TEPITOPE software.

TABLE 9

p33 peptides selected by the TEPITOPE program.
The CT and C72 peptide are also included for
reference.

| | | Position of | |
|---|---|---|---|
| | | peptide in | Peptide |
| Peptide | Amino Acid Sequence | p42 | Length |
| CT | LKPLAGVYRSLKKQIEK | 23-38 | 17 |
| CT-2 | VIYLKPLAGVYRSLKKQIE | 19-37 | 19 |
| p33-2 | FNLNLNDILNSRLK | 43-56 | 14 |
| p33-3 | LMQFKHISSNEYIIEDS | 69-85 | 17 |
| p33-4 | FLPFLTNIETLYNNLVNKID | 170-189 | 20 |
| p33-5 | LYNNLVNKIDDYLINLKAKIND | 180-201 | 22 |
| p33-6 | YLINLKAKIND | 191-201 | 11 |
| C72 | AHVKITKLSDLKAID | 209-223 | 15 |
| p33-7 | LVQNFPNTIISKLIEGK | 259-275 | 17 |
| p19-1 | FQDMLNISQHQCVKK | 276-292 | 15 |

To gain greater confidence in the predictive value of the TEPITOPE software to predict relevant T-cell epitopes, two known examples were run through the analysis. First, the tetanus toxin protein, which has been previously determined to have two well known universal T-cell epitopes, P2 and P30, was analyzed (Demontz et al, 1989). Both epitopes were successfully predicted by the software. In the second example, the ability to predict a universal T-cell epitope has been identified in the P. falciparum circumsporozoite protein (CSP) was assessed (amino acids 326-345 of the NF54 strain, Calvo-Calle et al, 1997). Again the program was successful at identifying this epitope in CSP.

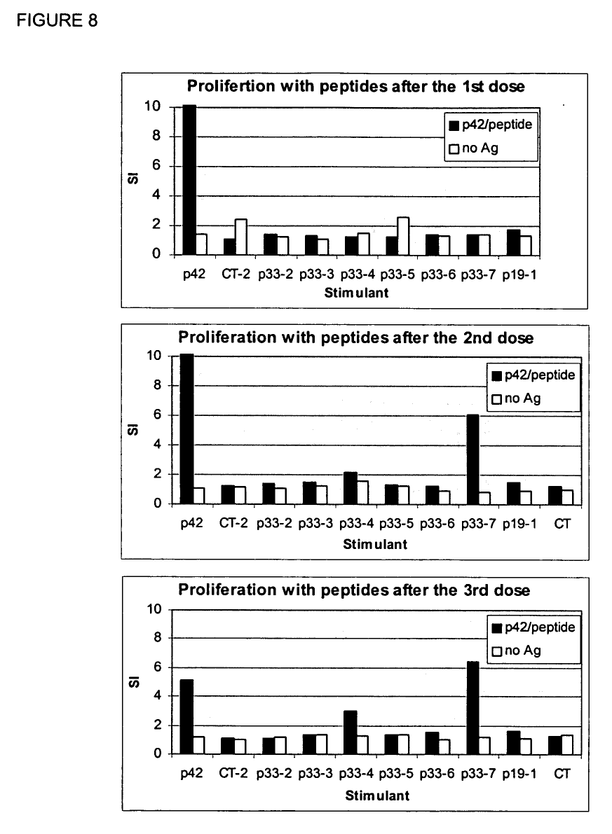

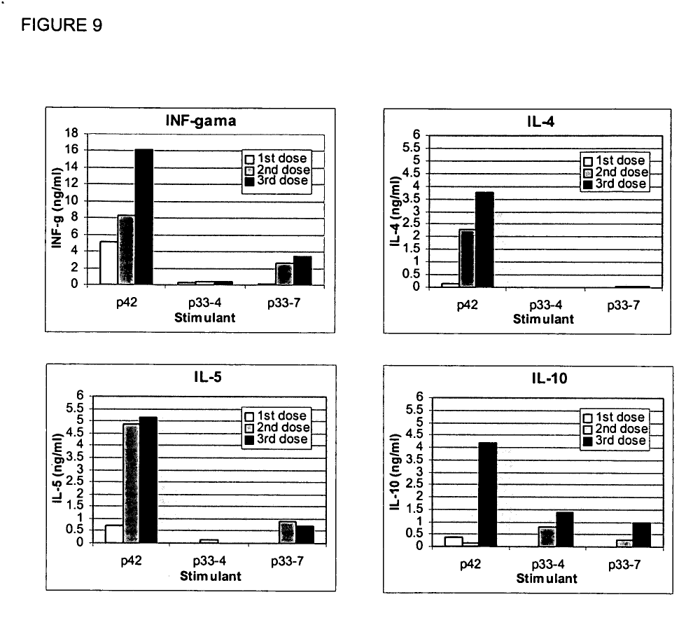

Despite the fact that the TEPITOPE program is based on the prediction of human HLA-DR epitopes, the utility of these predicted epitopes in mice has also been established. The epitopes in both the tetanus toxin and CSP examples used to test the program have also been demonstrated to elicit T-cell responses in mice. Therefore, mice can be used as a first step to evaluate these predicted epitopes. The following mouse experiment was designed to evaluate the potential of the peptides predicted by the TEPITOPE program for the p33 region. To test the potential of the TEPITOPE-predicted p33 peptides, Balb/c mice were immunized with 3 doses of MSP-1 p42 subunit and splenocytes were prepared from mice following each dose and tested for proliferation and cytokine secretion following stimulation with p42 or with p33 peptides. Two groups of 18 mice were utilized. One group received 10 μg doses of p42 formulated with 10 μg RC529 and ISA51 as previously described and the second group received adjuvant+ PBS and no p42 antigen. Following each dose six mice from each group were sacrificed and their spleens removed. Splenocytes were stimulated with MSP-l p42 subunit antigen at 5 μg/ml or peptide at 10 μg/ml. Proliferation results are shown in FIG. 8 for each dose and cytokine secretion data is shown in FIG. 9. Stimulation with MSP-1 p42 subunit resulted in a strong response after each dose. No proliferation was detected for the p33 peptides following the first dose. Following the second dose one peptide, p33-7, resulted in an SI value of 6.1. After the third dose, peptide p33-7 again resulted in an SI value of 6.4. Peptide p33-4 also resulted in a positive proliferation response, with an SI value of 3.0. Neither peptide CT-2 nor CT resulted in a proliferative response with the p42 primed splenocytes. Since only the MSP-1 p42 subunit and p33-4 and p33-7 peptides resulted in proliferative responses, cytokine secretion was only measured from the respective cultures.

The results of the cytokine expression are shown in FIG. 9. As with the proliferation results, the p42 subunit resulted in the highest levels of cytokines for all four cytokines tested. For each cytokine there was a general trend of levels increasing with each dose. The INF-gamma levels were the highest for the MSP-1 p42 subunit stimulated cells. For the p33-4 peptide, only moderate levels of IL-10 were detected. Moderate levels of INF-gamma, IL-5 and IL-10, but not IL-4, were detected for the p33-7 peptide. As with p42, the INF-gamma levels were the highest.

Peptide p33-4, FLPFLTNIETLYNNLVNKID (amino acid 170 to 189 of p42 or 1496 to 1515 of 3D7 MSP-1), is located 19 residues upstream on the N-terminus of the C72 region. It is one of three overlapping peptides (p33-4, p33-5, p33-6). Peptide p33-7, LVQNFPNTIISKLIEGK (amino acids 259 to 276 of p42 or 1585 to 1601 of 3D7 MSP-1), is located just upstream of the p19/p33 junction. There are five residues between the last residue of the p33-4 peptide and the first residue of p19. This p33-4 peptide is at the C-terminal end of the C72 region.

### Example 10

### Evaluation of the Non-Inhibitory Antibodies Elicited by Immunization with MSP-1 p42 Subunit Protein

In human seroepidemiological studies, protection against malaria has been shown to be mediated by antibodies directed against the C-terminal region of MSP-1 (Riley et al, 1992; Shai et al, 1995; Al-Yamen et al, 1996; and Shi et al, 1996). It has also been demonstrated that vaccine efficacy correlates with the ability of the antibodies to inhibit parasite growth in vitro (Chang et al, 1996; Hui and Siddiqui, 1987). In vitro studies with monoclonal antibodies and with antibodies from malaria-exposed humans have demonstrated the presence of non-parasite-inhibiting anti-MSP-1 p19 antibodies having the ability to block the effects of inhibitory anti-MSP-1 p 19 antibodies when both of them are combined (Guevara Patino et al, 1997; Holder et al, 1999; Nwuba et al, 2002; Uthaipibull et al, 2001). The term “blocking antibodies” is defined herein as antibodies that are capable of binding to MSP-1 C-terminal proteins and, when combined with antibodies known to inhibit the growth of parasites in vitro, block inhibitory activity of the other antibodies. Whether blocking antibodies are induced in formulated MSP-1 vaccines and what potential impact these antibodies may have on the overall efficacy has not been thoroughly investigated.

Immunization with MSP-1 p42 formulations has been shown to primarily induce anti-MSP-1 p19 antibodies (Hui et al, 1994; Kaslow et al, 1994). This raises the concern that active vaccination may induce blocking antibodies. This scenario is more detrimental than a MSP-1 vaccine that has no efficacy since the resulting immune response may interfere with subsequent vaccine attempts to induce protective anti-MSP-1 immunity. To test this possibility, the effects of non-inhibitory anti-MSP-1 p42 sera on the activities of anti-MSP-1 p42 sera that are known to strongly inhibit parasite growth were investigated. The hypothesis was that in the non-inhibitory anti-MSP-1 p42 sera, blocking antibodies may constitute a significant proportion of the responses, thereby interfering with the ability of inhibitory antibodies to kill parasites. Thus, these non-inhibitory sera may similarly interfere with other inhibitory anti-MSP-1 42 sera when they are mixed together.

The anti-MSP-1 p42 sera that were tested were from rabbits that were immunized with multiple doses of recombinant MSP-1 p42 subunit proteins as described in Examples 3 and 5. While many of the rabbits immunized with various formulations produced parasite inhibitory antibodies, this was not the case for all of the rabbits. To obtain a set of “non-inhibitory” sera, sera was selected from those rabbits that had good anti-p42 responses as determined by ELISA, but had less than 40% inhibitory antibodies.

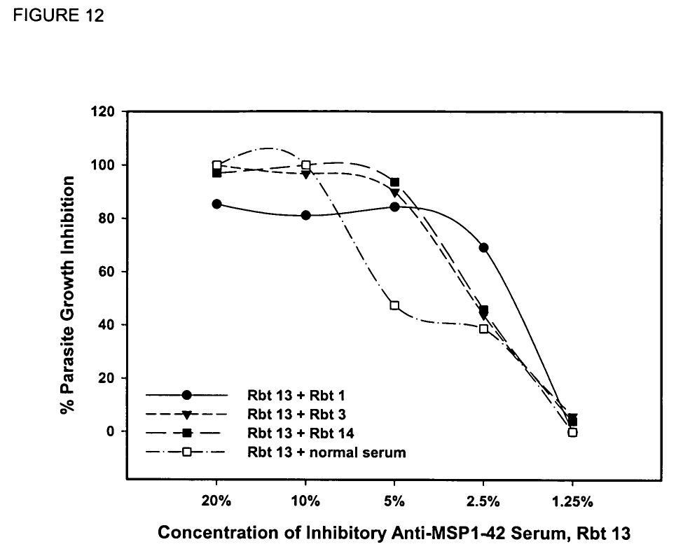

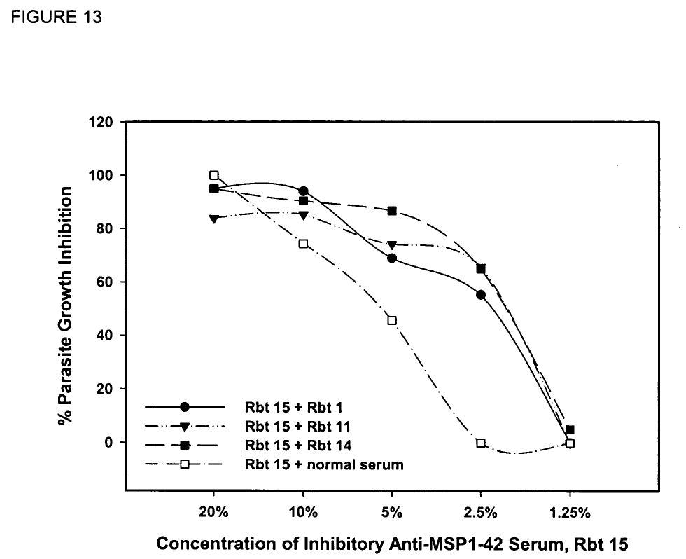

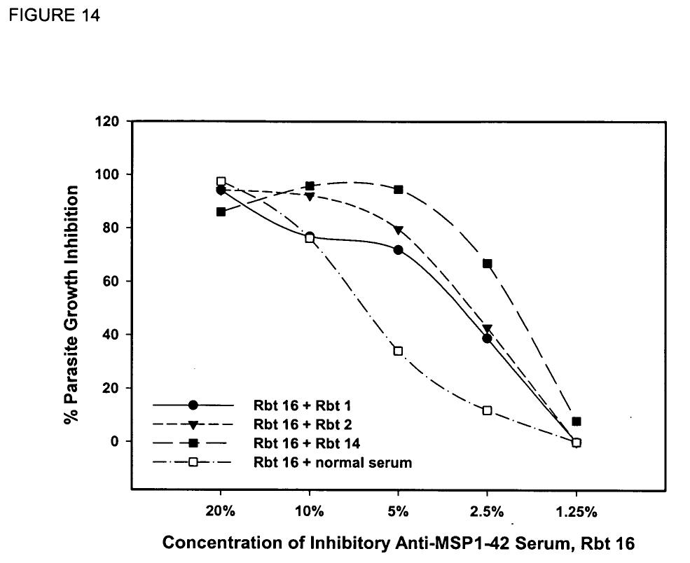

For the studies described in this Example, anti-MSP-1 p42 sera were divided into two sets and evaluated. In the first set, three sera (Rbt 13, 15, 16) were strongly parasite growth inhibitory (greater than 80%); whereas in the second set, five sera (Rbt 1, 2, 3, 11, 14) were non-inhibitory (see Table 10). To evaluate the effects of non-inhibitory anti-MSP-1 p42 sera on the activity of the three inhibitory sera, non-inhibitory sera were used to reconstitute the stepwise diluted inhibitory sera such that the final total serum concentration remained 25% by volume. As negative controls, pooled normal rabbit sera were similarly used for reconstitution. In vitro parasite inhibition assays were performed using sorbitol synchronized parasites as described in Example 3. FIG. 12 shows the parasite growth inhibitory activities of the individual serum from Rbt 13 supplemented by non-inhibitory sera from Rbts 1, 3, 14, and normal serum, respectively. FIG. 13 shows the parasite growth inhibitory activities of the individual serum from Rbt 15 supplemented by non-inhibitory sera from Rbts 1, 11, 14, and normal serum, respectively. FIG. 14 shows the parasite growth inhibitory activities of the individual serum from Rbt 16 supplemented by non-inhibitory sera from Rbts 1, 2, 14, and normal serum, respectively. In the groups supplemented by normal serum (“negative control groups”, shown as open squares in FIGS. 12-14), the potency of the growth inhibitory sera steadily diminished as serum concentrations fell, and became ineffective below 5%.

TABLE 10
In vitro Parasite Growth Inhibition Values of Selected
Anti-MSP-1 p42 Rabbit Sera.

| | |
|---|---|
| Rabbit (Rbt) # | % Parasite Growth Inhibition^{a} |

| | | |
|---|---|---|
| Rbt 13 | 100% | 100% |
| Rbt 15 | 97.6% | 100% |
| Rbt 16 | 92.9% | 96.6% |
| Rbt 1 | 0% | 36.0% |
| Rbt 2 | 0% | 0% |
| Rbt 3 | 0% | 0% |
| Rbt 11 | 23.2% | 17.9% |
| Rbt 14 | 28.8% | 24.2% |

^{a}Results of two independent assays shown.

These results are consistent with previous studies (Hui and Siddiqui, 1987). Two key observations are made. First, the addition of non-inhibitory sera did not have any detrimental effects on the ability of the inhibitory anti-MSP-1 p42 sera to inhibit parasites at various concentrations, i.e., no reduction of percentage inhibition across all test concentrations. Second, and most importantly, addition of non-inhibitory anti-MSP-1 42 sera was able to enhance the activities of the inhibitory anti-MSP-1 p42 sera, particularly at lower concentration ranges. This effect was specific since reconstitution of normal sera showed no significant enhancement.

While previous studies demonstrated the presence of blocking antibodies to MSP-1 (Guevara Patino et al, 1997; Nwuba et al, 2002; Uthaipibull et al, 2001), there is little information regarding the specific conditions in which these antibodies are preferentially induced. The immunization of rabbits with FUP MSP-1 p42 subunit and various adjuvants did not always result in the production of specific antibodies capable of inhibiting parasite growth despite high overall antibody titers as determined by ELISA. As shown in Example 5, in some cases the immunization of the rabbits resulted in the production of antibodies directed at MSP-1 p42; however, these antibodies are not capable of inhibiting parasite growth in vitro. The current studies demonstrate that these non-inhibitory sera have no detrimental effects on the activities of inhibitory anti-MSP-1 p42 sera, i.e., the non-inhibitory sera are non-blocking. These results are significant in that these non-inhibitory antibodies are able to enhance the efficiency of parasite inhibition when combined with low concentrations of inhibitory serum. Previous studies indicate that there are more than one critical epitope on MSP-1 p42 or MSP-1 p19 which are the targets of protective and/or inhibitory antibodies (Chappel and Holder, 1993; Ling et al, 1995; Morgan et al, 2004; Uthaipibull et al, 2001). Based on the results if the current studies, it is likely that the specificities of the non-inhibiting anti-MSP-1 p42 sera are different from those of the inhibitory sera. Since the antibody specificity of the reconstituted sera is likely to differ from those of the inhibitory sera (e.g., Rbt 13, 15, 16), it strongly suggests that hyper-immunization of rabbits with MSP-1 p42 results in a spectrum of specificities, i.e., no one serum contains the broadest possible specificity in terms of biological activities. It is possible that some of the relevant epitopes are inherently sub-dominant in either groups of rabbits, and thus could not efficiently elicit an appreciable antibody response.

Those skilled in the art also will readily appreciate that many modifications to the invention are possible within the scope of the invention. Accordingly, the scope of the invention is not intended to be limited to the preferred embodiments described above, but only by the claims appended to the non-provisional application incorporating this application.

### REFERENCES

Al-Yaman, F., B. Genton, K. J. Kramer, S. P. Chang, G. S. Hui, M. Baisor, and M. P. Alpers. 1996. Assessment of the role of naturally acquired antibody levels to Plasmodium falciparum merozoite surface protein-1 in protecting Papua New Guinean children from malaria morbidity. Am. J. Trop. Med. Hyg. 54:443-448.
Aucouturier J, Dupuis L, Deville S, Ascarateil S, Ganne V. 2002. Montanide ISA 720 and 51: a new generation of water in oil emulsions as adjuvants for human vaccines. Expert Rev Vaccines. 1(1):111-8.
Ballas, Z, J, et al, “Divergent Therapeutic and Immunologic Effects of Oligonucleotides with Distinct CpG Motifs”, J. of Immunology (2001) 167:4878-4886.
Bernard, A. R., T. A. Kost, L. Overton, C. Cavegn, J. Young, M. Bertrand, Z. Yahia-cherif, C. Chabert and A. Mills. 1992. Recombinant protein expression in Drosophila cell line:

comparison with the baculovirus system. Cytotechnology 15:139-144. 1994.
Bin L., S. Tsing, A. H. Kosaka, B. Nguyen, E. G. Osen, C. Bach, H. Chan and J. Barnett. 1996. Expression of human dopamine β-hydroxylase in Drosophila Schneider 2 cells. Biochem. J. 313:57-64.
Blackman M. J., H.-G. Heidrich, S. Donachie, J. S. McBride and A. A. Holder. 1990. A signle fragment of a malaria merozoite surface protein remains on the parasite during red blood cell invasion and is the target of invasion-inhibiting antibodies. J. Exp. Medicine 172:379-382.
Blackman M. J., I. T. Ling, S. C. Nicholls and A. A. Holder. 1991. Proteolytic processing of the Plasmodium falciparum merozoite surface protein-I produces a membrane bound fratgment contianing two epidermal gowth factor-like domains. Mol. Biochem. Parasitology 49:29-34.
Blackman M. J., and A. A. Holder. 1992. Secondary processing of the Plasmodium falciparum merozoite surface protein-1 by a calcium dependent membrane bound serine protese: shedding of MSP-1-33 as a noncovalently associated complex with other fragments of MSP-1. Mol. Biochem. Parasitology 50:307-316.
Burghaus P A, Wellde B T, Hall T, Richards R L, Egan A F, Riley E M, Ballou W R, Holder A A. 1996. Immunization of Aotus nancymai with recombinant C terminus of Plasmodium falciparum merozoite surface protein 1 in liposomes and alum adjuvant does not induce protection against a challenge infection. Infect Immun. 64(9):3614-9.
Burns J M Jr, Daly T M, Vaidya A B, Long C A. 1988. The 3′ portion of the gene for a Plasmodium yoelii merozoite surface antigen encodes the epitope recognized by a protective monoclonal antibody. Proc Natl Acad Sci USA. 85(2):602-6.
Calvo-Calle J M, Hammer J, Sinigaglia F, Clavijo P, Moya-Castro Z R, Nardin E H. 1997. Binding of malaria T cell epitopes to DR and DQ molecules in vitro correlates with immunogenicity in vivo: identification of a universal T cell epitope in the Plasmodium falciparum circumsporozoite protein. J Immunol. August 1997
Cavanagh D R, Dobano C, Elhassan I M, Marsh K, Elhassan A, Hviid L, Khalil E A, Theander T G, Arnot D E, McBride J S. 2001. Differential patterns of human immunoglobulin G subclass responses to distinct regions of a single protein, the merozoite surface protein 1 of Plasmodium falciparum. Infect Immun. 69(2):1207 11.
Chappel, J. A., and A. A. Holder. 1993. Monoclonal antibodies that inhibit Plasmodium falciparum invasion in vitro recognize the first growth factor-like domain of merozoite surface protein-1. Mol. Biochem. Parasitology. 60:303-312.
Chang, S. P., H. L. Gibson, C. T. Lee-Ng, P. J. Barr and G. S. N. Hui. 1992. A carboxyl-terminal fragment of Plasmodium falciparum gp195 expressed by recombinant baculovirus induces antibodies that completely inhibit parasite growth. J. Immuno. 149:548-555.
Chang. S. P, S. E. Case, W. L. Gosnell, A. Hashimoto, K. J. Kramer, L. Q. Tam, C. Q. Hashiro, C. M. Nikaido, H. L. Gibson, C. T. Lee-Ng, P. J. Barr, B. T. Yokata, and G. S. N. Hui. 1996. A recombinant 42-kilodalton C-terminal fragment of Plasmosium falciparum merozoite surface protein 1 protects Aotus monkeys against malaria. Infect. Immun. 64:253-261.
Cheung, A., J. Leban, A. R. Shaw, B. Merkli, J. Stocker, C. Chizolini, C. Sander and L. H. Perrin. 1986. Immunization with synthetic peptides of a Plasmodium falciparum surface anitgen induces anti-merozoite antibodies. PNAS 83:8328-8332.
Chu, R. S., et al, “CpG Oligodeoxynucleotides Act as Adjuvants that Switch on T Helper 1 (Th1) Immunity”, J. Exp. Med. (1997) 186:1623.
Cox J C and Coulter A R. 1997. Adjuvants—a classification and review of their modes of action. Vaccine February;15(3):248-56.Culp, J. S., H. Johansen, B. Hellmig, J. Beck, T. J. Matthews, A. Delers, and M. Rosenberg. 1991. Regulated expression allows high level production and secretion of HIV-1 gp120 envelope glycoprotein in Drosophila Schneider cells. Biotechnology 9:173-177.
Daly T M and Long C A. 1996. Influence of adjuvants on protection induced by a recombinant fusion protein against malarial infection. Infect Immun. 64:2602-8.
Darko C A, Angov E, Collins W E, Bergmann-Leitner E S, Girouard A S, Hitt S L, McBride J S, Diggs C L, Holder A A, Long C A, Barnwell J W, Lyon J A. 2005. The clinical-grade 42-kilodalton fragment of merozoite surface protein 1 of Plasmodium falciparum strain FVO expressed in Escherichia coli protects Aotus nancymai against challenge with homologous erythrocytic-stage parasites. Infect Immun. 73(1):287-97.
Demotz S, Lanzavecchia A, Eisel U, Niemann H, Widmann C, Corradin G. 1989. Delineation of several DR-restricted tetanus toxin T cell epitopes. J Immunol. Jan. 15;142(2):394-402.
De Souza J B, Ling I T, Ogun S A, Holder A A, Playfair J H. 1996. Cytokines and antibody subclass associated with protective immunity against blood-stage malaria in mice vaccinated with the C terminus of merozoite surface protein 1 plus a novel adjuvant. Infect Immun. 64(9):3532-6.
Egan, A. F., J. Morris, G. Barnish, S. Allen, B. M. Greenwood, D. C. Kaslow, A. A. Holder, and E. M. Riley. 1996. Clinical immunity to Plasmodium falciparum malaria is associated with serum antibodies to the 19-kDa C-terminal fragment of the merozoite surface antigen, PfMSP-1. J. Infect. Dis. 173:765-769.
Etlinger, H. M., P. Caspers, H. Matile, H-J Schoenfeld, D. Stueber and B. Takacs. 1991. Ability of recombinant or native proteins to protect monkeys against heterologous challenge with Plasmodium falciparum. Infect. Immunity 59:3498-3503.
Evans J T, Cluff C W, Johnson D A, Lacy M J, Persing D H, Baldridge J R. 2003. Enhancement of antigen-specific immunity via the TLR4 ligands MPL adjuvant and Ribi.529. Expert Rev Vaccines. 2(2):219-29.
Good, M. F., D. C. Kaslow, and L. H. Miller. 1998. Pathway and strategies for developing a malaria blood-stage vaccine. Ann. Rev. Immunonol. 16:57-87.
Guevara Patino, J. A., A. A. Holder, J. S. McBride, and M. J. Blackman. 1997. Antibodies that inhibit malaria merozoite surface protein-I processing and erythrocyte invasion are blocked by naturally acquired human antibodies. J Exp. Med. 186:1689-1699.
Gupta R K, and Siber G R. 1995. Adjuvants for human vaccines—current status, problems and future prospects. Vaccine. 13(14):1263-76.
Hall, R., A. Osland, J. E. Hyde, D. L. Simmons, I. A. Hope and J. G. Scaife. 1984. Processing polymorphism, and biological significance of P190, a major surface antigen of the erthrocytic forms of Plasmodium falciparum. Mol. Biol. Parisitology 11:61-81.
Hartmann, G. and Krieg, A., “Mechanism and Function of a Newly Identified CpG DNA Motif in Human Primary B Cells.” J. Immunol. (2000) 164:944-952.
Hartmann, G., et al, “Delineation of a CpG Phosphorothioate Oligonucleotide for Activating Primate Immune Responses in vitro and in vivo”, J. of Immunol. (2000) 164:1617-1624.
Herrera, S., M. A. Herrera, B. L. Berlaza, Y. Burki, P. Caspers, H. Dobeli, D. Rotman and U. Certa. 1990. Immunization of Aotus monkeys with Plasmodium falciparum blood-stage recombinant proteins. PNAS 87:4017-4021.
Hirunpetcharat C, Tian J H, Kaslow D C, van Rooijen N, Kumar S, Berzofsky J A, Miller L H, Good M F. 1997. Complete protective immunity induced in mice by immunization with the 19-kilodalton carboxyl-terminal fragment of the merozoite surface protein-1 (MSP1[19]) of Plasmodium yoelii expressed in Saccharomyces cerevisiae: correlation of protection with antigen-specific antibody titer, but not with effector CD4+ T cells. J Immunol. 159(7):3400-11.
Holder, A. A., J. S. Sandhu, Y. Hillman, L. S. Davey, S. C. Nicholls, H. Cooper and M. J. Lockyer. 1987. Processing of the precursor of the major merozoite surface antigens of Plasmodium falciparum. Parsitology 94:199-208.
Holder, A. A., R. R. Freeman and S. C. Nicholls. 1988. Immunization against Plasmodium falciparum with recombinant polypeptides produced in Escherichia coli. Parasite Immunology 10:607-617.
Hui, G. S. and W. A. Siddiqui. 1987. Serum from Pf195 protected Aotus monkeys inhibit Plasmodium falciparum growth in vitro. Exp. Parasitol. 64:519-522.
Hui G S. 1994. Liposomes, muramyl dipeptide derivatives, and nontoxic lipid A derivatives as adjuvants for human malaria vaccines. Am J Trop Med Hyg. 50(4 Suppl):41-51.
Incardona, J. P. and T. L. Rosenberry. 1996. Construction and characterization of secreted and chimeric transmembrane forms of Drosophila acetylcholinesterase: a large truncation of the C-terminal signal peptide does not eliminate glycoinositol phospholipid anchoring. Mol. Biol. of the Cell 7:595-611.
Ivey-Hoyle M, Culp J S, Chaikin M A, Hellmig B D, Matthews T J, Sweet R W, Rosenberg M. 1991. Envelope glycoproteins from biologically diverse isolates of immunodeficiency viruses have widely different affinities for CD4. Proc Natl Acad Sci U S A. Jan. 15;88(2):512-6.
Kaslow, D. C., G. Hui, and S. Kumar. 1994. Expression and antigenicity of Plasmodium falciparum major merozoite surface protein (MSP1(19)) variants secreted from Saccharomyces cerevisiae. Mol. Biochem. Parasitol. 63:283-289.
Krieg, A. M. et al, “CpG Motifs in Bacterial DNA Trigger Direct B-Cell Activation”, Nature (1995) 374:546.
Kumar, S., A. Yadava, D. B. Keister, J. H. Tian, M. Ohl, K. A. Perdue-Greenfield, L. H. Miller and D. C. Kaslow. 1995. Immunogenicity and in vivo efficacy of recombinant Plasmodium falciparum merzoite surface protein-1 in Aotus monkeys. Molecular Medicine 1:325-332.
Kumar, S., W. Collins, A. Egan, A. Yadava, O. Garraud, M. J. Blackman, J. A. Guevara Patino, C. Diggs and D. C. Kaslow. 2000. Immunogenicity and efficacy in Aotus monkeys of four recombinant Plasmodium falciparum vaccines in multiple adjuvant formulations based on the 19-kilodalton C terminus of merozoite surface protein 1. Infect Immun 68:2215-2223.
Ling I T, Ogun S A, Momin P, Richards R L, Garcon N, Cohen J, Ballou W R, Holder A A. 1997. Immunization against the murine malaria parasite Plasmodium yoelii using a recombinant protein with adjuvants developed for clinical use. Vaccine. 15(14):1562-7.
Lyon, J. A., R. H. Geller, J. D. Haynes, J. D. Chulay, and J. L Weber. 1986. Epitope map and processing scheme for the 195,000-dalton surface glycoprotein of Plasmodium falciparum merozoites deduced from cloned overlapping segments of the gene. PNAS 83:2989-2993.
Margalit H, Spouge J L, Cornette J L, Cease K B, Delisi C, Berzofsky J A. 1987. Prediction of immunodominant helper T cell antigenic sites from the primary sequence. J Immunol. Apr. 1;138(7):2213-29.
Mcbride, J. S., and H-G. Heidrich. 1987. Fragments of the polymorphic Mᵣ 185,000 glycoprotein from the surface of isolated Plasmodium falciparum merozoites from an antigenic complex. Mol. Biochem. Parasitology. 23:71-84.
Melquist, J. L., L. Kasturi, S. L. Spitalnik, and S. H. Shakin-Eshelman. 1998. The amino acid following an Asn-X-Ser/Thr sequon is an important determinant of N-linked core glycosylation efficiency. Biochemistry. 37:6833-6837.
Menendez-Arias L, Rodriguez R. 1990. A BASIC microcomputer program for prediction of B and T cell epitopes in proteins. Comput Appl Biosci. April;6(2):101-5.
Modis Y, Ogata S, Clements D, and Harrison S C. 2003. A ligand-binding pocket in the dengue virus envelope glycoprotein. Proc Natl Acad Sci U S A. 100(12):6986-91
Morgan, W. D., M. J. Lock, T. A. Frenkiel, M. Grainger, and A. A. Holder. 2004. Malaria parasite-inhibitory antibody epitopes on Plasmodium falciparum merozoite surface protein-1(19) mapped by TROSY NMR. Mol. Biochem. Parasitol. 138:29-36.
Nwuba, R. I., O. Sodeinde, C. I. Anumudu, Y. O. Omosun, A. B. Odaibo, A. A. Holder, and M. Nwagwu. 2002. The human immune response to Plasmodium falciparum includes both antibodies that inhibit merozoite surface protein 1 secondary processing and blocking antibodies. Infect. Immun. 70:5328-5331.
Patarroyo, M. E., P. Romero, M. L. Torres, P. Clavijo, A. Moreno, A. Martinez, R. Rodriguez, F. Guzman and E. Cabezas. 1987. Induction of protective immunity against experimental infection with malaria using synthetic peptides. Nature 328:629-632.
Perera K L, Handunnetti S M, Holm I, Longacre S, Mendis K. 1998. Baculovirus merozoite surface protein 1 C-terminal recombinant antigens are highly protective in a natural primate model for human Plasmodium vivax malaria. Infect Immun. 66(4):1500-6.
Pirson, P. J. and M. E. Perkins. 1985. Characterization with monoclonal antibodies of a surface antigen of Plasmodium falciparum merozoites. J. Immunology 134:1946-1951.
Riley, E. M., S. J. Allen, J. G. Wheeler, M. J. Blackman, S. Bennett, B. Takacs, H. J. Schonfeld, A. A. Holder, and B. M. Greenwood. 1992. Naturally acquired cellular and humoral immune responses to the major merozoite surface protein-1 (PfMSP-1) of the Plasmodium falciparum are associated with malria morbidity. Parasite Immunol 14:321-337.
Schneider, I. 1972. Cell Lines Derived from Late Embryonic Stages of Drosophila melanogaster. J. Embryol. Exp. Morphol. 27:353-356.
Shai, S., M. J. Blackman, and A. A. Holder. 1995. Epitopes in the 19 kD fragment of the Plasmodium falciparum major merozoite surface protein-1 (PfMSP-1) recognized by human antibodies. Parasite Immunology 17:269-275.
Shi, Y. P., U. Sayed, S. H. Qari, J. M. Roberts, V. Udhayakumar, A. J. Oloo, W. A. Hawley, D. C. Kaslow, B. L. Nahlen, And A. A. Lal. 1996. Natural immune response to the C-terminal 19-kilodalton domain of Plasmodium falciparum merozoite surface protein 1. Infect. Immun. 64:2716-2723.
Siddiqui, W. A., L. Q. Tam, S-C Kan, K. J. Kramer, S. E. Case, K. L. Palmer, K. M. Yamaga, and G. S. N. Hui. 1986. Induction of protective immunity to monoclonal-antibody-defined Plasmodium falciparum antigens requires strong adjuvants in Aotus monkeys. Infect. Immun. 52(1):314-318.
Siddiqui, W. A., L. Q. Tam, K. J. Kramer, G. S. N. Hui, S. E. Case, K. M. Yamaga, S. P. Chang, E. B. T. Chan and S-C Kan. 1987. Merozoite surface coat precursor protein completely protects Aotus monkeys against Plasmodium falciparum malaria. PNAS 84:3014-3018.
Singh H, Raghava G P. 2001. ProPred: prediction of HLA-DR binding sites. Bioinformatics. December;17(12):1236-7.
Stowers A W, Miller L H. 2001. Are trials in New World monkeys on the critical path for blood-stage malaria vaccine development? Trends Parasitol. 17(9):415-9.
Stowers A W, Cioce V, Shimp R L, Lawson M, Hui G, Muratova O, Kaslow D C, Robinson R, Long C A, Miller L H. 2001. Efficacy of two alternate vaccines based on Plasmodium falciparum merozoite surface protein 1 in an Aotus challenge trial. Infect Immun. 69(3):1536-46.
Sturniolo T, Bono E, Ding J, Raddrizzani L, Tuereci O, Sahin U, Braxenthaler M, Gallazzi F, Protti M P, Sinigaglia F, Hammer J. 1999. Generation of tissue-specific and promiscuous HLA ligand databases using DNA microarrays and virtual HLA class II matrices. Nat Biotechnol. June 1999;17(6):555-61.
Udhayakumar V, Anyona D, Kariuki S, Shi Y P, Bloland P B, Branch O H, Weiss W, Nahlen B L, Kaslow D C, and Lal A A. 1995. Identification of T and B cell epitopes recognized by humans in the C terminal 42 kDa domain of the Plasmodium falciparum merozoite surface protein (MSP) 1. J Immunol. 154(11):6022 30.
Ulrich, J T and Myers K R. 1995. in Vaccine Design: The Subunit and Adjuvant Approach, ed. Powell, M F and Newman, M J. Plenum Press, NY.
Uthaipibull, C., B. Aufiero, S. E. Syed, B. Hansen, J. A. Patino, E. Angov, I. T. Ling, K. Fegeding, W. D. Morgan, C. Ockenhouse, B. Birdsall, J. Feeney, J. A. Lyon, and A. A. Holder. 2001. Inhibitory and Blocking Monoclonal Antibody Epitopes on Merozoite Surface Protein 1 of the Malaria Parasite Plasmodium falciparum. J. Mol. Biol. 307:1381-1394.
Van der Straten, A., H. Johansen, M. Rosenberg and R. W. Sweet. 1989. Introduction and constitutive expression of gene products in cultures Drosophila cells using hygromycin B selection. Methods in Mol. and Cell. Biol. 1:1-8.
Weeratna, R. D. et al., “CpG DNA induces stronger immune responses with less toxicity than other adjuvants”, Vaccine (2000) 18:1755-1762.
World Health Organization. 1997. World malaria situation in 1994. Wkly Epidemiol Rec 72:269-290.
Yang, C. W. E. Collins, J. S. Sullivan, D. C. Kaslow, L. Xiao, and A. A. Lal. 1999. Partial protection against Plasmodium vivax blood-stage infection in Saimiri monkeys by immunization with a recombinant C-terminal fragment of merozoite surface protein 1 in block copolymer adjuvant. Infect Immun. 67(1):342-9.

What is claimed is:

1. A method for producing a recombinant subunit malaria vaccine comprising:
expressing and secreting from stably transformed insect cells a recombinant Plasmodium protein subunit, wherein the protein subunit is derived from Plasmodium merozoite surface protein 1 and represents a carboxy-terminal portion of a native p42 protein; and
formulating said recombinant subunit protein with one or more adjuvants to produce an immunogenic composition that induces the production of Plasmodium parasite inhibition antibodies in a host vaccinated with the immunogenic composition.

2. The method of claim 1, wherein the species of Plasmodium is selected from the group consisting of falciparum, vivax, malariae, and ovale.

3. The method of claim 1, where in the carboxy-terminal portion of a native p42 protein has an amino acid sequence selected from the group consisting of SEQ ID NO:1, SEQ ID NO:2, and SEQ ID NO:3.

4. The method of claim 1, wherein said expression is from stably transformed Drosophila melanogaster S2 cells.

5. The method of claim 1, wherein the one or more adjuvants are selected from the group consisting of a Montanide adjuvant alone or a Montanide adjuvant in combination with a monophosphoryl lipid A derived adjuvant.

6. The method of claim 5, wherein said Montanide adjuvant is ISA51.

7. The method of claim 5, wherein said Montanide adjuvant is ISA720.

8. The method of claim 5, wherein the monophosphoryl lipid A derived adjuvant is RC529.

9. The method of claim 1, wherein the recombinant Plasmodium protein subunit is an N-terminally truncated MSP-1 p42 protein having an amino acid sequence selected from the group consisting of SEQ ID NO:7, SEQ ID NO:8, and SEQ ID NO:9.

10. The method of claim 1, wherein the carboxy-terminal protein subunit portion of the Plasmodium merozoite surface protein 1 is purified by immuno-affinity chromatography.

11. An immunogenic composition comprising a recombinant, carboxy-terminal protein subunit of the Plasmodium merozoite surface protein 1 and one or more adjuvants, wherein the protein subunit is produced by expression in, and secretion from, stably transformed insect cells.

12. The immunogenic composition of claim 11, wherein the species of Plasmodium providing the merozoite surface protein 1 is selected from the group consisting of falciparum, vivax, malariae, and ovale.

13. The immunogenic composition of claim 11, wherein said the stably transformed insect cells are Drosophila melanogaster S2 cells.

14. The immunogenic composition of claim 11, wherein the one or more adjuvants are selected from the group consisting of a Montanide adjuvant alone or a Montanide adjuvant in combination with a monophosphoryl lipid A derived adjuvant.

15. The immunogenic composition of claim 14, wherein said Montanide adjuvant is ISA51.

16. The immunogenic composition of claim 14, wherein said Montanide adjuvant is ISA720.

17. The immunogenic composition of claim 14, wherein the monophosphoryl lipid A derived adjuvant is RC529.

18. The immunogenic composition of claim 11, wherein the recombinant, carboxy-terminal protein subunit portion of the Plasmodium merozoite surface protein 1 is purified by immuno-affinity chromatography.

19. The immunogenic composition of claim 11, wherein the recombinant protein is an MSP-1 C-terminal subunit protein having an amino acid sequence selected from the group consisting of SEQ ID No: 1, SEQ ID No: 2, SEQ ID No: 3, SEQ ID NO:7, SEQ ID NO:8, and SEQ ID NO:9.

20. The amino acid of claim 19, wherein the amino acid sequence consists of a amino acid sequence with at least 95% sequence identity to the selected amino acid sequence.

21. The amino acid sequence of claim 19, wherein the amino acid sequence consists of an amino acid sequence with at least 90% sequence identity to the selected amino acid sequence.

22. The immunogenic composition of claim 11, 12, 13, 14, 15, 16, 17, 18, 19, 20, or 21, wherein the immunogenic composition is administered to a subject in a vaccine.

23. The immunogenic composition of claim 11, 12, 13, 14, 15, 16, 17, 18, 19, 20, or 21, wherein the immunogenic composition is administered to a subject in a vaccine and the immunogenic composition produces inhibitory antibodies, with or without enhancing antibodies, in the subject.

24. An immunogenic composition comprising a recombinant, carboxy-terminal protein subunit of the Plasmodium merozoite surface protein 1 and one or more adjuvants, wherein the one or more adjuvants are selected from the group consisting of a Montanide adjuvant alone or a Montanide adjuvant in combination with a monophosphoryl lipid A derived adjuvant, wherein the protein subunit is produced by expression in, and secretion from, stably transformed insect cells, wherein the protein subunit has an amino acid sequence selected from the group consisting of SEQ ID No: 1, SEQ ID No: 2, SEQ ID No: 3, SEQ ID NO:7, SEQ ID NO:8, and SEQ ID NO:9, and wherein the subunit protein is purified by immuno-affinity chromatography.


SEQUENCE LISTING (12 sequences)

SEQ ID NO: 1 - protein, 373 aa, Plasmodium falciparum
AISVTMDNILSGFENEYDVIYLKPLAGVYRSLKKQIEKNIFTFNLNLNDILNSRLKKRKYFLDVLESDLMQFKHISSNEYIIEDSFKLLNSEQKNTLLKSYKYIKESVENDIKFAQEGISYYEKVLAKYKDDLESIKKVIKEEKEKFPSSPPTTPPSPAKTDEQKKESKFLPFLTNIETLYNNLVNKIDDYLINLKAKINDCNVEKDEAHVKITKLSDLKAIDDKIDLFKNHNDFDAIKKLINDDTKKDMLGKLLSTGLVQNFPNTIISKLIEGKFQDMLNISQHQCVKKQCPENSGCFRHLDEREECKCLLNYKQEGDKCVENPNPTCNENNGGCDADAKCTEEDSGSNGKKITCECTKPDSYPLFDGIFCS

SEQ ID NO: 2 - protein, 373 aa, Plasmodium falciparum
AISVTMDNILSGFENEYDVIYLKPLAGVYRSLKKQIEKNIFTFNLNLNDILNSRLKKRKYFLDVLESDLMQFKHISSNEYIIEDSFKLLNSEQKNTLLKSYKYIKESVENDIKFAQEGISYYEKVLAKYKDDLESIKKVIKEEKEKFPSSPPTTPPSPAKTDEQKKESKFLPFLTNIETLYNNLVNKIDDYLINLKAKINDCNVEKDEAHVKITKLSDLKAIDDKIDLFKNPYDFEAIKKLINDDTKKDMLGKLLSTGLVQNFPNTIISKLIEGKFQDMLNISQHQCVKKQCPENSGCFRHLDEREECKCLLNYKQEGDKCVENPNPTCNENNGGCDADATCTEEDSGSSRKKITCECTKPDSYPLFDGIFCS

SEQ ID NO: 3 - protein, 355 aa, Plasmodium falciparum
AVTPSVIDNILSKIENEYEVLYLKPLAGVYRSLKKQLENNVMTFNVNVKDILNSRFNKRENFKNVLESDLIPYKDLTSSNYVVKDPYKFLNKEKRDKFLSSYNYIKDSIDTDINFANDVLGYYKILSEKYKSDLDSIKKYINDKQGENEKYLPFLNNIETLYKTVNDKIDLFVIHLEAKVLNYTYEKSNVEVKIKELNYLKTIQDKLADFKKNNNFVGIADLSTDYNHNNLLTKFLSTGMVFENLAKTVLSNLLDGNLQGMLNISQHQCVKKQCPQNSGCFRHLDEREECKCLLNYKQEGDKCVENPNPTCNENNGGCDADAKCTEEDSGSNGKKITCECTKPDSYPLFDGIFCS

SEQ ID NO: 4 - DNA, 1119 bp, Plasmodium falciparum
gcaatatctgtcacaatggataatatcctctcaggatttgaaaatgaatatgatgttatatatttaaaacctttagctggagtatatagaagcttaaaaaaacaaattgaaaaaaacatttttacatttaatttaaatttgaacgatatcttaaattcacgtcttaagaaacgaaaatatttcttagatgtattagaatctgatttaatgcaatttaaacatatatcctcaaatgaatacattattgaagattcatttaaattattgaattcagaacaaaaaaacacacttttaaaaagttacaaatatataaaagaatcagtagaaaatgatattaaatttgcacaggaaggtataagttattatgaaaaggttttagcgaaatataaggatgatttagaatcaattaaaaaagttatcaaagaagaaaaggagaagttcccatcatcaccaccaacaacacctccgtcaccagcaaaaacagacgaacaaaagaaggaaagtaagttccttccatttttaacaaacattgagaccttatacaataacttagttaataaaattgacgattacttaattaacttaaaggcaaagattaacgattgtaatgttgaaaaagatgaagcacatgttaaaataactaaacttagtgatttaaaagcaattgatgacaaaatagatctttttaaaaaccataacgacttcgaagcaattaaaaaattgataaatgatgatacgaaaaaagatatgcttggcaaattacttagtacaggattagttcaaaattttcctaatacaataatatcaaaattaattgaaggaaaattccaagatatgttaaacatttcacaacaccaatgcgtaaaaaaacaatgtccagaaaattctggatgtttcagacatttagatgaaagagaagaatgtaaatgtttattaaattacaaacaagaaggtgataaatgtgttgaaaatccaaatcctacttgtaacgaaaataatggtggatgtgatgcagatgccaaatgtaccgaagaagattcaggtagcaacggaaagaaaatcacatgtgaatgtactaaacctgattcttatccacttttcgatggtattttctgcagt

SEQ ID NO: 5 - DNA, 1119 bp, Plasmodium falciparum
gcaatatctgtcacaatggataatatcctctcaggatttgaaaatgaatatgatgttatatatttaaaacctttagctggagtatatagaagcttaaaaaaacaaattgaaaaaaacatttttacatttaatttaaatttgaacgatatcttaaattcacgtcttaagaaacgaaaatatttcttagatgtattagaatctgatttaatgcaatttaaacatatatcctcaaatgaatacattattgaagattcatttaaattattgaattcagaacaaaaaaacacacttttaaaaagttacaaatatataaaagaatcagtagaaaatgatattaaatttgcacaggaaggtataagttattatgaaaaggttttagcgaaatataaggatgatttagaatcaattaaaaaagttatcaaagaagaaaaggagaagttcccatcatcaccaccaacaacacctccgtcaccagcaaaaacagacgaacaaaagaaggaaagtaagttccttccatttttaacaaacattgagaccttatacaataacttagttaataaaattgacgattacttaattaacttaaaggcaaagattaacgattgtaatgttgaaaaagatgaagcacatgttaaaataactaaacttagtgatttaaaagcaattgatgacaaaatagatctttttaaaaacccttacgacttcgaagcaattaaaaaattgataaatgatgatacgaaaaaagatatgcttggcaaattacttagtacaggattagttcaaaattttcctaatacaataatatcaaaattaattgaaggaaaattccaagatatgttaaacatttcacaacaccaatgcgtaaaaaaacaatgtccagaaaattctggatgtttcagacatttagatgaaagagaagaatgtaaatgtttattaaattacaaacaagaaggtgataaatgtgttgaaaatccaaatcctacttgtaacgaaaataatggtggatgtgatgcagatgccacatgtaccgaagaagattcaggtagcagcagaaagaaaatcacatgtgaatgtactaaacctgattcttatccacttttcgatggtattttctgcagt

SEQ ID NO: 6 - DNA, 1065 bp, Plasmodium falciparum
gcagtaactccttccgtaattgataacatactttctaaaattgaaaatgaatatgaggttttatatttaaaacctttagcaggtgtttatagaagtttaaaaaaacaattagaaaataacgttatgacatttaatgttaatgttaaggatattttaaattcacgatttaataaacgtgaaaatttcaaaaatgttttagaatcagatttaattccatataaagatttaacatcaagtaattatgttgtcaaagatccatataaatttcttaataaagaaaaaagagataaattcttaagcagttataattatattaaggattcaatagatacggatataaattttgcaaatgatgttcttggatattataaaatattatccgaaaaatataaatcagatttagattcaattaaaaaatatatcaacgacaaacaaggtgaaaatgagaaataccttccctttttaaacaatattgagaccttatataaaacagttaatgataaaattgatttatttgtaattcatttagaagcaaaagttctaaattatacatatgagaaatcaaacgtagaagttaaaataaaagaacttaattacttaaaaacaattcaagacaaattggcagattttaaaaaaaataacaatttcgttggaattgctgatttatcaacagattataaccataataacttattgacaaagttccttagtacaggtatggtttttgaaaatcttgctaaaaccgttttatctaatttacttgatggaaacttgcaaggtatgttaaacatttcacaacaccaatgcgtaaaaaaacaatgtccacaaaattctggatgtttcagacatttagatgaaagagaagaatgtaaatgtttattaaattacaaacaagaaggtgataaatgtgttgaaaatccaaatcctacttgtaacgaaaataatggtggatgtgatgcagatgccaaatgtaccgaagaagattcaggtagcaacggaaagaaaatcacatgtgaatgtactaaacctgattcttatccacttttcgatggtattttctgcagt

SEQ ID NO: 7 - protein, 133 aa, Plasmodium falciparum
GARSGGGGTMLGKLLSTGLVQNFPNTIISKLIEGKFQDMLNISQHQCVKKQCPENSGCFRHLDEREECKCLLNYKQEGDKCVENPNPTCNENNGGCDADAKCTEEDSGSNGKKITCECTKPDSYPLFDGIFCS

SEQ ID NO: 8 - protein, 174 aa, Plasmodium falciparum
GARSGGGGTAHVKITKLSDLKAIDDKIDLFKNPYDFEAIKKLINDDTKKDMLGKLLSTGLVQNFPNTIISKLIEGKFQDMLNISQHQCVKKQCPENSGCFRHLDEREECKCLLNYKQEGDKCVENPNPTCNENNGGCDADATCTEEDSGSSRKKITCECTKPDSYPLFDGIFCS

SEQ ID NO: 9 - protein, 191 aa, Plasmodium falciparum
GARSLKPLAGVYRSLKKQIEKGGGGTAHVKITKLSDLKAIDDKIDLFKNPYDFEAIKKLINDDTKKDMLGKLLSTGLVQNFPNTIISKLIEGKFQDMLNISQHQCVKKQCPENSGCFRHLDEREECKCLLNYKQEGDKCVENPNPTCNENNGGCDADATCTEEDSGSSRKKITCECTKPDSYPLFDGIFCS

SEQ ID NO: 10 - DNA, 399 bp, Plasmodium falciparum
ggcgcccgctccggaggaggcggaaccatgctgggtaagctactaagcaccggactggtgcagaacttcccgaatacgatcatctcgaagctgatcgagggtaagttccaggacatgctaaatatttcccagcaccagtgcgtcaagaagcagtgccccgagaactcgggttgcttccgccacctagatgagcgtgaggagtgtaagtgcctcctgaattacaagcaagagggcgacaagtgcgtcgaaaatcccaaccccacgtgcaacgagaacaacggcggctgcgatgccgatgccaagtgcaccgaggaggatagcggcagcaatggcaagaagatcacctgcgagtgcacgaagcccgacagctatcccctattcgatggcatcttctgctcg

SEQ ID NO: 11 - DNA, 522 bp, Plasmodium falciparum
ggagccagatctggtggcggtggtaccgcacatgttaaaataactaaacttagtgatttaaaagcaattgatgacaaaatagatctttttaaaaacccttacgacttcgaagcaattaaaaaattgataaatgatgatacgaaaaaagatatgcttggcaaattacttagtacaggattagttcaaaattttcctaatacaataatatcaaaattaattgaaggaaaattccaagatatgttaaacatttcacaacaccaatgcgtaaaaaaacaatgtccagaaaattctggatgtttcagacatttagatgaaagagaagaatgtaaatgtttattaaattacaaacaagaaggtgataaatgtgttgaaaatccaaatcctacttgtaacgaaaataatggtggatgtgatgcagatgccacatgtaccgaagaagattcaggtagcagcagaaagaaaatcacatgtgaatgtactaaacctgattcttatccacttttcgatggtattttctgcagt

SEQ ID NO: 12 - DNA, 573 bp, Plasmodium falciparum
ggagccagatctttaaaacctttagctggagtatatagaagcttaaaaaaacaaattgaaaaaggtggcggtggtaccgcacatgttaaaataactaaacttagtgatttaaaagcaattgatgacaaaatagatctttttaaaaacccttacgacttcgaagcaattaaaaaattgataaatgatgatacgaaaaaagatatgcttggcaaattacttagtacaggattagttcaaaattttcctaatacaataatatcaaaattaattgaaggaaaattccaagatatgttaaacatttcacaacaccaatgcgtaaaaaaacaatgtccagaaaattctggatgtttcagacatttagatgaaagagaagaatgtaaatgtttattaaattacaaacaagaaggtgataaatgtgttgaaaatccaaatcctacttgtaacgaaaataatggtggatgtgatgcagatgccacatgtaccgaagaagattcaggtagcagcagaaagaaaatcacatgtgaatgtactaaacctgattcttatccacttttcgatggtattttctgcagt